SEQUENCE LISTING (25 sequences)

SEQ ID NO: 1 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 3 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 4 - DNA, 18 bp, synthetic construct
  misc_feature 1..18: note = oligonucleotide primer Il10 reverse
  source 1..18: mol_type = other DNA
cagactcaatacacactg

SEQ ID NO: 5 - DNA, 20 bp, synthetic construct
  misc_feature 1..20: note = oligonucleotide primer Il33 forward
  source 1..20: mol_type = other DNA
atgggaagaagctgatggtg

SEQ ID NO: 6 - DNA, 20 bp, synthetic construct
  misc_feature 1..20: note = oligonucleotide primer Il33 reverse
  source 1..20: mol_type = other DNA
ccgaggactttttgtgaagg

SEQ ID NO: 7 - DNA, 20 bp, synthetic construct
  misc_feature 1..20: note = oligonucleotide primer Ucp1 forward
  source 1..20: mol_type = other DNA
ggcctctacgactcagtcca

SEQ ID NO: 8 - DNA, 20 bp, synthetic construct
  misc_feature 1..20: note = oligonucleotide primer Ucp1 reverse
  source 1..20: mol_type = other DNA
taagccggctgagatcttgt

SEQ ID NO: 9 - DNA, 22 bp, synthetic construct
  misc_feature 1..22: note = oligonucleotide primer Ppargc1a forward
  source 1..22: mol_type = other DNA
agccgtgaccactgacaacgag

SEQ ID NO: 10 - DNA, 23 bp, synthetic construct
  misc_feature 1..23: note = oligonucleotide primer Ppargc1a reverse
  source 1..23: mol_type = other DNA
gctgcatggttctgagtgctaag

SEQ ID NO: 11 - DNA, 20 bp, synthetic construct
  misc_feature 1..20: note = oligonucleotide primer Dio2 forward
  source 1..20: mol_type = other DNA
tgccaccttcttgactttgc

SEQ ID NO: 12 - DNA, 20 bp, synthetic construct
  misc_feature 1..20: note = oligonucleotide primer Dio2 reverse
  source 1..20: mol_type = other DNA
ggttccggtgcttcttaacc

SEQ ID NO: 13 - DNA, 22 bp, synthetic construct
  misc_feature 1..22: note = oligonucleotide primer Cox7a1 forward
  source 1..22: mol_type = other DNA
aaaccgtgtggcagagaagcag

SEQ ID NO: 14 - DNA, 23 bp, synthetic construct
  misc_feature 1..23: note = oligonucleotide primer Cox7a1 reverse
  source 1..23: mol_type = other DNA
cccaagcagtataagcagtaggc

SEQ ID NO: 15 - DNA, 22 bp, synthetic construct
  misc_feature 1..22: note = oligonucleotide primer Adrb3 forward
  source 1..22: mol_type = other DNA
aactgaaacagcagacagggac

SEQ ID NO: 16 - DNA, 20 bp, synthetic construct
  misc_feature 1..20: note = oligonucleotide primer Adrb3 reverse
  source 1..20: mol_type = other DNA
cccccatgtacaccctagtt

SEQ ID NO: 17 - DNA, 19 bp, synthetic construct
  misc_feature 1..19: note = oligonucleotide primer Th forward
  source 1..19: mol_type = other DNA
ccaaggttcattggacggc

SEQ ID NO: 18 - DNA, 20 bp, synthetic construct
  misc_feature 1..20: note = oligonucleotide primer Th reverse
  source 1..20: mol_type = other DNA
ctctcctcgaataccacagc

SEQ ID NO: 19 - DNA, 22 bp, synthetic construct
  misc_feature 1..22: note = oligonucleotide primer Lipe forward
  source 1..22: mol_type = other DNA
gctcatctcctatgacctacgg

SEQ ID NO: 20 - DNA, 22 bp, synthetic construct
  misc_feature 1..22: note = oligonucleotide primer Lipe reverse
  source 1..22: mol_type = other DNA
tccgtggatgtgaacaaccagg

SEQ ID NO: 21 - DNA, 22 bp, synthetic construct
  misc_feature 1..22: note = oligonucleotide primer Pnpla2 forward
  source 1..22: mol_type = other DNA
ggaaccaaaggacctgatgacc

SEQ ID NO: 22 - DNA, 22 bp, synthetic construct
  misc_feature 1..22: note = oligonucleotide primer Pnpla2 reverse
  source 1..22: mol_type = other DNA
acatcaggcagccactccaaca

SEQ ID NO: 23 - DNA, 24 bp, synthetic construct
  misc_feature 1..24: note = oligonucleotide primer Tbp forward
  source 1..24: mol_type = other DNA
ctaccgtgaatcttggctgtaaac

SEQ ID NO: 24 - DNA, 22 bp, synthetic construct
  misc_feature 1..22: note = oligonucleotide primer Tbp reverse
  source 1..22: mol_type = other DNA
aatcaacgcagttgtccgtggc

SEQ ID NO: 25 - DNA, 20 bp, synthetic construct
  misc_feature 1..20: note = oligonucleotide primer Il10 forward
  source 1..20: mol_type = other DNA
aataagctccaagaccaagg

(19) United States
(12) Patent Application Publication
(10) Pub. No.: US 2023/0158112 A1
(43) Pub. Date: May 25, 2023

(54) Targeting gamma-delta T Cells in Obesity and Cachexia

(71) Applicant: The Brigham and Women's Hospital, Inc., Boston, MA (US)

(72) Inventors: Michael Brenner, Fredrick, MD (US); Lydia Lynch, Newton Highlands, MA (US); Ayano Kohlgruber, Brighton, MA (US)

(21) Appl. No.: 17/929,209

(22) Filed: Sep. 1, 2022

Related U.S. Application Data
(63) Continuation of application No. 16/609,098, filed on Oct. 28, 2019, now abandoned.
(60) Provisional application No. 62/491,753, filed on Apr. 28, 2017.

(51) Int. Cl. A61K 38/19; A61P 3/04; A61K 38/17; A61K 38/20; A61K 39/395; C07K 16/24

(52) U.S. Cl. CPC A61K 38/191; A61P 3/04; A61K 38/1793; A61K 38/20; A61K 39/3955; C07K 16/244; C07K 2317/76

(57) ABSTRACT

Methods of promoting or inhibiting weight loss by modulating activity or levels of γδ T cells.

### CLAIM OF PRIORITY

This application is a continuation of U.S. patent application Ser. No. 16/609,098, filed Oct. 28, 2019, which is a §371 National Stage Application of PCT/US2018/030153, filed Apr. 30, 2018, which claims priority under 35 USC §119(e) to U.S. Patent Application Ser. No. 62/491,753, filed on Apr. 28, 2017. The entire contents of the foregoing are hereby incorporated by reference.

### FEDERALLY SPONSORED RESEARCH OR DEVELOPMENT

This invention was made with government support under Grant Nos. AI11304603 and AI113046 awarded by the National Institutes of Health. The government has certain rights in the invention.

### SEQUENCE LISTING

This application contains a Sequence Listing that has been submitted electronically as an XML file named “29618-0163002_SL_ST26.XML.”The XML file, created on Sep. 1, 2022, is 32,427 bytes in size. The material in the XML file is hereby incorporated by reference in its entirety.

### TECHNICAL FIELD

Described herein are methods of promoting or inhibiting weight loss by modulating activity or levels of γδ T cells.

### BACKGROUND

Adipose tissue harbors a unique immune compartment that is important for physiologic responses to fasting and feeding, regulation of body weight, and thermogenesis. Compared to lymphoid organs, approximately 80-90% of the adipose immune system is innate. Much of what we know about the adipose tissue immune system suggests its major roles are not focused on fighting infection. Instead, obesity studies reveal that perturbations in immune cells or signaling molecules can either protect from or contribute to inflammation and insulin sensitivity. Although less understood, the resident innate immune compartment of adipose tissue also likely has important functions in the absence of obesity.

Besides innate myeloid cells, a substantial component of adipose tissue is comprised of innate lymphocytes such as type 2 innate lymphoid cells (ILC2s), invariant natural killer T (iNKT) cells, mucosal-associated invariant T (MAIT) cells, natural killer (NK) cells, and γδ T cells^{1,2}. In the lean state, ILC2s and iNKT cells are critical for maintaining an anti-inflammatory environment through secretion of type 2 cytokines that support the function and survival of eosinophils, alternatively activated macrophages (AAM), and regulatory T (T_{reg}) cells³⁻⁶. Moreover, under other lo conditions including cold challenge, ILC2s and iNKT cells can induce thermogenic programs in adipose tissue^{7,8}. Conversely, in obesity, NK^{8,9} cells and MAIT¹⁰⁻¹² cells secrete pro-inflammatory cytokines that impair glucose handling by adipocytes, hepatocytes and muscle cells and interfere with insulin production and insulin signaling. Despite recent advances in our understanding of adipose innate lymphocytes, the role of γδ T cells in this dynamic organ remains largely unknown.

### SUMMARY

γδ T cells are situated at barrier sites and guard the body from infection and damage. However, little is known about their role(s) outside of host defense in non-barrier tissues. Here, we characterize a highly enriched, tissue-resident population of γδ T cells in adipose tissue that regulates age-dependent regulatory T (T_{reg}) cell expansion and controls core body temperature in response to environmental fluctuations. Mechanistically, innate PLZF⁺ γδ T cells produced tumor necrosis factor (TNF) and interleukin-17A (IL-17A) and determined PDGFRα⁺ and Pdpn⁺ stromal cell production of IL-33 in adipose tissue. Mice lacking γδ T cells or IL-17A exhibited reductions in both ST2⁺ T_{reg} cells and IL-33 abundance in visceral adipose tissue. Remarkably, these mice also lack the ability to regulate core body temperature at thermoneutrality and after cold challenge. Together, these findings support the methods of targeting resident γδ T cells in adipose tissue to alter immune homeostasis and body temperature control, to reduce obesity or to treat cachexia.

Thus, provided herein are methods for promoting weight loss. The methods can include administering to a subject in need thereof therapeutically effective amounts of a combination of agents comprising: (i) Tumor Necrosis Factor alpha (TNFα) or an agonist of the TNF Receptor (TNFR), and (ii) Interleukin 17 (IL-17) or IL-33. Alternatively or in addition, e.g., as an alternative to or in addition to either or both of the previously listed agents, the methods can include administration of an agent that activates γδ T cells, e.g., an activating monoclonal antibody against a specific T cell receptor (TCR), e.g., a monoclonal antibody targeting Vd2, a number of which are available commercially, pyrophosphates or butyrophilins. In some embodiments, the subject has a body mass index (BMI) of 29 or above. In some embodiments, the agents are modified to increase half-life. In some embodiments, the agents are targeted to adipose tissue.

Also provided herein are methods of inhibiting weight loss (or promoting lo weight gain). The methods include administering to a subject in need thereof therapeutically effective amounts of a combination of agents comprising: (i) an inhibitor of Tumor Necrosis Factor alpha (TNFα) or the TNF Receptor (TNFR), and (ii) an inhibitor of Interleukin 17 (IL-17), IL-17 Receptor, or IL-33.

Further, provided herein are methods for inhibiting weight loss that include administering to a subject in need thereof a therapeutically effective amount of an agent comprising an inhibitory anti-IL-33 antibody.

In some embodiments, the subject has a chronic illness or cancer. In some embodiments, the cancer is not IL-17 dependent, e.g., wherein IL-17 is not present in significant levels in the tumor. In some embodiments, the cancer is pancreatic cancer.

In some embodiments, the subject has COPD, diabetic kidney disease, or heart failure.

In some embodiments, the agents are modified to increase half-life. In some embodiments, the agents are targeted to adipose tissue.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1D. γδ T cells are enriched and resident in adipose tissue. (A) Representative flow cytometry plots of γδ T cells from eWAT SVF. (B) Frequency of γδ T cells across various adipose tissue depots as a percentage of CDR3_{ε}⁺ T cells in male eWAT (n≥4 mice per group). (C) Flow cytometry of lymphocytes in blood, liver, spleen, and adipose tissue of CD45.1⁺ and CD45.2⁺ congenic C57BL/6 parabiotic pairs joined at 6 wks of age and analyzed 2 wks later (left); and frequency lo pie charts of CD45.1⁺ and CD45.2⁺ CD8⁺ and γδ T cells (right). (D) Frequency of Vδ1⁺, Vδ2⁺, and Vδ3⁺ γδ T cells (% of CD45⁺ PI⁻ cells) in peripheral blood (PBMCs) or from matched omental fat from patients before bariatric surgery. SVF, stromal vascular fraction. eWAT, epididymal white adipose tissue. Each symbol represents an individual mouse; small horizontal lines indicate the mean. Data are representative of three experiments (A,B,D; mean±s.e.m. in B) or one experiment (C).

FIGS. 2A-2G. PLZF discriminates two γδ T cell populations. (A) Representative flow cytometry and frequency quantification of CD3_{ε}^{hi} and CD3_{ε}^{lo} γδ T cells from eWAT SVF across adipose, liver, and spleen (n=5 mice). (B) CD27 expression by CD3_{ε}^{hi} and CD3_{ε}^{lo} γδ T cells (left) and subset quantification from adipose, liver, and spleen (right) (n=5 mice). (C) Representative histograms of mean fluorescence intensity (MFI) of CD69, CD44, CD127, and CD45RB expression by CD3_{ε}^{hi} and CD3_{ε}^{lo} γδ T cells. (D) Representative histogram of PLZF expression by CD3_{ε}^{hi} and CD3_{ε}^{lo} γδ T cells (left) and MFI quantification across adipose, liver, and spleen (right) (n=5 mice). (E) Representative flow cytometry of TCRβ⁺ versus TCRδ⁺ cells of PLZF⁺CD45⁺ cells from eWAT SVF. (F) Immunofluorescence microscopy of whole-mount adipose tissue from Zbtb16GFP mice injected with dextran (grey line in bottom left quarter). Scale bar, 100 μm. (G) Frequency of γδ T cells from wild-type (WT) and Zbtb16^{-/-} mice (n=5). NS, not significant (P>0.05); * P<0.05 and **** P<0.0001 (One-way ANOVA). Data are representative of three experiments (A,B,C,D,E; mean±s.e.m. in A,D) or two experiments (F,G; mean±s.e.m. in G).

FIGS. 3A-3F. γδ T cells are important for adipose T_{reg} accumulation.

Numbers per gram eWAT of γδ T and Foxp3⁺ T_{reg} cells (A) and ILC2s and iNKT cells (B) at 5, 8, 11, 21 and 28 wks of age (n≥4 mice per timepoint). (C) Ratio (log₂ normalized fold change) of CD8⁺ T, NK, CD4⁺ T, ILC2s, and T_{reg} cell numbers from Tcrd^{-/-} mice compared to WT mice (left) at 16 wks (n=10 mice). Frequency and numbers of Foxp3⁺ T_{reg} cells in eWAT between WT and Tcrd^{-/-} mice (right) at 16 wks (n=4 mice per group). (D) Quantitative real-time PCR for Il10 expression normalized to Tbp from sorted CD25⁺CD4⁺ T_{reg} cells from WT and Tcrd^{-/-} mice at 16 wks (n=5 mice). (E) Frequency (left) and MFI (right) of cell surface KLRG1 expression on eWAT Foxp3⁺ T_{reg} cells at 16 wks (n=5 mice). (F) Representative flow cytometry of ST2⁺Foxp3⁺ T_{reg} cells from eWAT (left) and cell numbers at 5, 8, 11 and 22 wks of age between WT and Tcrd^{-/-} littermates (right) (n=5 mice). Each symbol represents an individual mouse; small horizontal lines indicate the mean. NS, not significant (P>0.05); * P<0.05; ** P<0.01; *** P<0.001. (Students t test in C,D,E; One-way ANOVA in F). Data are representative of three experiments (A,B,F; mean±s.e.m. in A,B,F) or two experiments (C,D,E; mean±s.e.m. in C,D,E).

FIGS. 4A-4D. PLZF⁺ γδ T cells are innate-IL17A producing cells. (A) Heatmap of top 60 genes differentially expressed (FDR-adjusted P value<0.01) between PLZF⁺ (left) and PLZF⁻ (right) γδ T cells. (B) Scatter plot of gene transcripts differentially expressed by PLZF⁺ and PLZF⁻ γδ T cells from 14 wk old male mice. (C) Flow cytometry of RORγt (top) and T-bet (bottom) expression in PLZF⁺ and PLZF⁻ γδ T cells from eWAT SVF. (D) Representative intracellular cytokine staining (top) and quantification (bottom) on gated γδ T cells from eWAT SVF for TNF, IL-17A and IFN-γ after 4 h stimulation with PMA and ionomycin (n=4 mice). NS, not significant (P>0.05); * P<0.05; ** P<0.01; **** P<0.0001. (One-way ANOVA in D). Data are representative of two experiments (C,D; mean±s.e.m. in D).

FIGS. 5A-5F. ST2⁺ T_{reg} numbers depend on PLZF⁺ γδ T cells and IL-17A. (A) Frequency of PLZF⁺ and PLZF⁻ γδ T cells from eWAT at 5, 8, 11 and 22 wks of age (n=5 mice per timepoint). (B) Numbers of GFP⁺ CD3_{ε}^{hi} and CD3_{ε}^{lo} γδ T cells from eWAT of Il17a^{GFP} male mice at 10, 13, 20 and 27 wks of age (n=5 mice per timepoint). (C) Numbers and frequency of ST2⁺ and total T_{reg} cells from eWAT of 16 wk old males from WT and Il17a^{-/-} mice (n=5 mice, pooled). (D) Representative flow cytometry of γδ T cells stained with anti-CD27, 17D1, anti-Vγ1 and anti-Vγ4 antibodies to characterize TCR usage. (E) Representative flow cytometry plots of γδ T cells from WT and Vg4/6^{-/-} eWAT (left) and quantification of CD3_{ε}^{hi} and CD3_{ε}^{lo} γδ frequencies (right) (n=5 mice). (F) Numbers and frequency of ST2⁺ and total T_{reg} cells from eWAT of 16 wk old WT and Vg4/6^{-/-} male mice (n≥4 mice). Each symbol represents an individual mouse; small horizontal lines indicate the mean. NS, not significant (P>0.05); * P<0.05 (One-way ANOVA in A,C,F). Data are pooled across two experiments (A,B,C,F; mean±s.e.m. in A,B,C,F) or representative of two experiments (D,E; mean±s.e.m. in E).

FIGS. 6A-6H. TNF and IL-17A induce IL-33 in adipose stromal cells. (a) IL-33 protein from SVF eWAT lysates of 5-8, 15-16 and 20+ wk WT male mice normalized to total SVF protein by ELISA (n=4 mice per timepoint). (B) IL-33 (left) to and IL-2 (right) protein from SVF eWAT and splenic lysates of 14 wk old WT and Tcrd^{-/-} male mice (n≥3 mice, pooled). (C) IL-33 protein from SVF eWAT lysates of 16 wk old WT, Vg4/6^{-/-} and Il17a^{-/-} male mice normalized to total SVF protein (n≥4 mice). (D) Representative flow cytometry plots for stromal cell subsets in eWAT SVF of male WT mice. (E) Cell surface IL-17R, CD26, CD9 and Cdh11 MFI of CD45⁻Pdpn^{hi}PDGFRα⁻ and CD45⁻Pdpn^{lo}PDGFRα⁺ stromal cells. (F) Quantitative real-time PCR for Il33 mRNA normalized to Tbp from sorted Pdpn^{hi}, PDGFRα⁺, CD31⁺, and CD45⁺ cells from WT mice (n=6). (G) 3T3L1 or primary adipose stromal cells derived from eWAT SVF were unstimulated (unstim) or stimulated with TNF^{lo} (0.1 ng/mL), TNF (1 ng/mL), IL-17A^{lo} (0.1 ng/mL), IL-17A (1 ng/mL), or TNF (1 ng/mL)+IL-17A (1 ng/mL) for 18 h. Il33 transcript levels were measured by quantitative real-time PCR (left) and protein levels measured by ELISA (right). (H) Primary human stromal cells derived from visceral (Lonza) and subcutaneous (ATCC) adipose tissues were unstimulated (unstim) or stimulated with TNF^{lo} (0.1 ng/mL), TNF (1 ng/mL), IL-17A^{lo} (0.1 ng/mL), IL-17A (1 ng/mL), or TNF (1 ng/mL)+IL-17A (1 ng/mL) for 18 h. Cell lysates were collected and IL-33 protein measured by ELISA. Each symbol represents an individual mouse; small horizontal lines indicate the mean. NS, not significant (P>0.05); * P<0.05; ** P<0.01; *** P<0.001; **** P<0.0001 (One-way ANOVA in A,B,C,F,G,H). Data are pooled across three experiments (A,B,C; mean±s.e.m. in A,B,C) or representative of two experiments (D,E,F,G,H; mean±s.e.m. in F,G,H).

FIGS. 7A-7F. γδ T cells are important for adaptive thermogenesis after cold. (A) Representative histology of hematoxylin- and eosin-stained BAT and lipid droplet quantification from WT, Tcrd^{-/-} and Vg4/6^{-/-} mice after 6 h at 4° C. Scale bars, 500 μm. (B) Immunoblot analysis and densitometry quantification of UCP1 and HSP90 in BAT of WT, Tcrd^{-/-} and Vg4/6^{-/-} mice after 6 h at 4° C. (n=5 mice). (C) Quantitative real-time PCR of thermogenesis genes from WT and Tcrd^{-/-} BAT (top), and WT and Vg4/6^{-/-} BAT (bottom) after 6 h at 4° C. (n≥5 mice). (D) Immunoblot analysis of pHSL and HSL and quantitative real-time PCR of Lipe (Hsl) from iWAT of WT, Tcrd^{-/-} and Vg4/6^{-/-} mice after 6 h at 4° C. (n≥5 mice). (E) Quantitative real-time PCR of thermogenesis genes from WT, Tcrd^{-/-} and Vg4/6^{-/-} iWAT after 6 h at 4° C. (n≥5 mice). (F) Body temperature (left) and energy expenditure (right) measured between WT and Tcrd^{-/-} male mice from 30° C. to 4° C. (n=5). Each symbol lo represents an individual mouse; small horizontal lines indicate the mean. Gene expression normalized to Tbp. Immunoblots have been cropped to show relevant proteins. NS, not significant (P>0.05); * P<0.05; ** P<0.01; *** P<0.001 (metabolic variable adjusted for differences in body composition by ANCOVA in F; One-way ANOVA in A,E,F; Students t test in B,C). Data are representative across two experiments (A-F; mean±s.e.m. in A-C,D-F).

FIGS. 8A-I. IL-17A promotes thermogenic responses in brown and inguinal adipose tissue. (A) Frequency of γδ T cells of CD45⁺ cells in BAT and iWAT 0, 8, 24 h at 4° C. (n=4 mice). (B) Frequency of IL-17A producing γδ T cells from BAT and iWAT after 5 h stimulation with PMA and ionomycin (n=10 mice). (C) Frequency of immune cells from BAT and iWAT that produce TNF, IL-17A, or TNF+IL-17A after 4 h stimulation with PMA and ionomycin at 4° C. (n=5 mice). (D) Representative histology of hematoxylin- and eosin-stained BAT and lipid droplet quantification from WT and Il17a^{-/-} mice after 6 h at 4° C. Scale bars, 500 μm. (E) Quantitative real-time PCR of Ucp1 (left) and immunoblot analysis of UCP1 and HSP90 (right) in BAT tissue obtained from WT and Il17a^{-/-} mice after 6 h at 4° C. (n≥5 mice). (F) Quantitative real-time PCR of Lipe (Hsl) from WT and Il17a^{-/-} iWAT after 6 h at 4° C. (n≥5 mice). (G) Quantitative real-time PCR of Ucp1 from WT and Il17a^{-/-} iWAT after 6 h at 4° C. (n>5 mice). (H) WT and Il17a^{-/-} mice were gradually shifted from 30° C. to 4° C. at a continuous rate and monitored for survival. Mice were rescued when body temperature dropped to <28° C. (n=5 mice). (I) Energy expenditure measured between WT and Il17a^{-/-} male mice (n=5 mice). Each symbol represents an individual mouse; small horizontal lines indicate the mean. Gene expression normalized to Tbp. Immunoblots have been cropped to show relevant proteins. NS, not significant (P>0.05); * P<0.05; ** P<0.01; *** P<0.001 (metabolic variable adjusted for differences in body composition by ANCOVA in H,I; One-way ANOVA in A; Students t test in D-G; Log-rank Mantel-Cox test in H). Data are representative across two experiments (A-G; mean±s.e.m. in A-G).

FIGS. 9A-9C. Immunophenotyping panels for adipose immune cell quantification. (A) Representative flow cytometry plots to identify ILC2s, γδ T, CD4⁺ T, Foxp3⁺ T_{reg} , and ST2⁺Foxp3⁺ T_{reg} cells. (B) Representative flow cytometry plots to identify eosinophils, B220⁺CD19⁺ B, CD19⁺ B, NK, iNKT, and CD8⁺ T cells. (C) Numbers of CD4⁺ T, CD8⁺ T, eosinophils, CD19⁺ B, B220⁺CD19⁺ B, and NK cells per gram of eWAT at 5, 8, 11, 21 and 28 wks of age in male mice (n=5, pooled). Each symbol represents an individual mouse; small horizontal lines indicate the mean. Data are representative across two experiments (A,B,C; mean±s.e.m. in c).

FIGS. 10A-10E. ILC2, iNKT, and T_{reg} numbers in IL-17A KO and Vγ4/6 KO mice. (A) Numbers (left) and frequency (right) of ILC2s in eWAT from WT, Vg4/6^{-/-} and Il17a^{-/-} 16 wk old mice (n=5, pooled). (B) Numbers (left) and frequency (right) of iNKTs in eWAT from WT, Vg4/6^{-/-} and Il17a^{-/-} 16 wk old mice (n=5, pooled). (C) Quantification of numbers (top) and frequencies (bottom) of T_{reg} cells and ST2+T_{reg} cells from spleen, lung, and adipose tissue from WT, Vg4/6^{-/-} and Il17a^{-/-} 16 wk old mice (n≥3). (D) IL-33 protein from SVF eWAT lysates of 11 wk male WT and mice normalized to total SVF protein by ELISA (n≥3). (E) Numbers (top) and frequency (bottom) of T_{reg} cells and ST2⁺ T_{reg} cells from WT and Il17a^{-/-} eWAT at 11 wks of age (n≥4). Each symbol represents an individual mouse; small horizontal lines indicate the mean. NS, not significant (P>0.05); * P<0.05; **** P<0.0001 (One-way ANOVA in A-C; Students t test in D-E). Data are pooled across two experiments (A-E; mean±s.e.m. in A-E).

FIGS. 11A-11D. In vitro and in vivo cytokine stimulations of epididymal adipose stromal cells. (A) 3T3L1 adipose fibroblasts were unstimulated (unstim) or stimulated with TNF^{lo} (0.1 ng/mL), TNF (1 ng/mL), IL-17A^{lo} (0.1 ng/mL), IL-17A^{hi} (1 ng/mL), IL-1β^{lo} (0.1 ng/mL), IL-1β^{hi} (1 ng/mL), (0.1 ng/mL), IFN-γ^{hi} (1 ng/mL), or a combination of the cytokines as indicated for 18 h. IL-33 protein was measured by ELISA. (B) WT mice were injected with saline or TNF (1 μg) and IL-17A (0.5 μg) every third day for a total of nine days and eWAT RNA isolated. Il33 transcript levels were measured by quantitative real-time PCR and normalized to Tbp (n≥5). Representative flow cytometry plots (C) and Il33 expression from iWAT stromal cells (D) after WT mice were injected with saline or TNF (1 μg) and IL-17A (0.5 μg) every third day for a total of nine days. Il33 normalized with Tbp (n≥3, pooled). Small horizontal lines indicate the mean. ** P<0.01; **** P<0.0001 (One-way ANOVA in A,D; Students t test in B). Data are pooled across two experiments run in triplicates (A; mean±s.e.m. in A). Data are representative of two experiments (B-D; mean±s.e.m. in B,D).

FIGS. 12A-B. Decreased numbers and not gene expression likely contributes to lower IL-33 protein. (A) Quantification of numbers (top) and frequencies (bottom) of CD31⁺, PDGFRα⁺Pdpn⁻, Pdpn^{hi}, and Pdpn^{lo} eWAT stromal cells from 23 wk old WT, Tcrd^{-/-}, Vg4/6^{-/-}, and Il17a^{-/-} male mice (n≥3 mice per genotype). (B) Quantitative real-time PCR for Il33 expression normalized with Tbp from sorted Pdpn^{hi}, PDGFR□⁺, CD31⁺, and CD45⁺ cells from WT, Tcrd^{-/-},Vg4/6^{-/-}, and Il17a^{-/-} mice (n≥3 mice per genotype). Each symbol represents an individual mouse; small horizontal lines indicate the mean. NS, not significant (P>0.05); * P<0.05; ** P<0.01; *** P<0.001; **** P<0.0001. (One-way ANOVA in A-B). Data are representative of two experiments (A-B; mean±s.e.m. in A-B).

FIGS. 13A-13B. γδT cells promote temperature regulation and IL-33 homeostasis in BAT and iWAT. (A) IL-33 protein was quantified from cell lysates of eWAT, iWAT, and BAT from WT, Tcrd^{-/-} and Vg4/6^{-/-} mice using ELISA (left). Quantitative real-time PCR for Il33 expression normalized with Tbp (right) from iWAT and BAT of WT, Tcrd^{-/-} and Vg4/6^{-/-} mice (n≥4). (B) Energy expenditure measured from WT and Tcrd^{-/-} mice injected with sterile saline at time 0 h and subsequently injected with selective β3-adrenergic receptor, CL-316 243, (1 mg/kg) at 3 h (n=5 per genotype). Small horizontal lines indicate the mean. NS, not significant; *P<0.05; ** P<0.01; *** P<0.001. (One-way ANOVA in A; Metabolic variable adjusted for differences in body composition by ANCOVA in B). Data are representative of two experiments (A; mean±s.e.m. in A) or one experiment (C; mean±s.e.m. in C).

FIGS. 14A-14E. IL-17A promotes thermogenic responses in BAT and iWAT. (A) Frequency (left) and numbers (right) of γδT cells at 0, 8, and 24 h at 4° C. in BAT and iWAT (n≥3 mice per condition). (B) Quantitative real-time PCR of Ppargc1a, Dio2, and Cox7a1 normalized to Tbp in BAT between WT and Il17a^{-/-} mice (n≥3). (C) Quantitative real-time PCR of Ppargc1a and Dio2 normalized to Tbp in iWAT between WT and Il17a^{-/-} mice (n>3). (D) Mice were gradually shifted from 30° C. to 4° C. at a continuous rate and body temperature measured between WT and Il17a^{-/-} male mice (n=5 mice per genotype). (E) Body temperature (top) and RER (bottom) measured for 72 h at thermoneutrality after acclimation between WT and Il17a^{-/-} male mice (n=5 per genotype). Each symbol represents an individual mouse; small horizontal lines indicate the mean. NS, not significant (P>0.05); * P<0.05; ** P<0.01; *** P<0.001. (Students t test in B-C; One-way ANOVA in A; Metabolic variable adjusted for differences in body composition by ANCOVA in D-E). Data are representative of two experiments (A-C; mean±s.e.m. in A-C).

FIGS. 15A-15B. Gene expression analysis of BAT and iWAT. Quantitative real-time PCR of Th, Adrb3, Lipe (Hsl), and Pnpla2 (Atgl) in brown (A) and inguinal (B) adipose tissue obtained from WT, Tcrd^{-/-}, Vg4/6^{-/-} and Il17a^{-/-} mice at room temperature (25° C.) and after 6 h cold at 4° C. Genes normalized to Tbp (n≥4 mice per condition). Each symbol represents an individual mouse; small horizontal lines indicate the mean. NS, not significant (P>0.05); * P<0.05; ** P<0.01. (One-way ANOVA in A,B). Data are representative of two experiments (A,B; mean±s.e.m. in A,B).

FIGS. 16A-16E. γδT cells directly and indirectly influence adaptive thermogenesis. (A) Differentiated brown adipocytes were stimulated with indicated amounts of TNF^{lo} (0.1 ng/mL), TNF^{hi} (1 ng/mL), IL-17A^{lo} (0.1 ng/mL), IL-17A^{hi} (1 ng/mL), for 18 h and Ucp1, Dio2, Cidea, and Il33 transcript levels were measured by quantitative real-time PCR and normalized with Tbp. (B) Differentiated brown adipocytes were stimulated with either IL-33^{lo} (10 ng/mL), IL-33^{hi} (100 ng/mL), and analyzed as in A. (C) Representative flow cytometry plots (left) of iWAT stromal cells after WT mice were injected with saline (top row) or TNF (1 μg) and IL-17A (0.5 μg) every third day for a total of nine days. Pdpn⁺PDGFRα⁻ and PDGFRα⁺iWAT stromal cells were sorted and gene expression of Ucp1, Ppargc1a, and Dio2 measured by quantitative real-time PCR and normalized with Tbp (n≥3). Frequency (top) and numbers (bottom) of eosinophils, ILC2s, iNKT, and T_{reg} cells from WT, Tcrd^{-/-} and Vg4/6^{-/-} brown (D) and inguinal (E) adipose tissue from 22 wk male mice (n≥4 mice per group). Each symbol represents an individual replicate or mouse. Data are representative of two experiments (A-E; mean±s.e.m. in A-E). NS, not significant (P>0.05); * P<0.05; ** P<0.01; *** P<0.001; **** P<0.0001. (One-way ANOVA in A-E).

### DETAILED DESCRIPTION

A key adaptive cell type in adipose tissue is the Foxp3⁺ T_{reg} cell. T_{reg} cells are low in numbers in adipose tissue of mice until 20 weeks of age, after which they greatly expand and comprise 40-80% of the CD4⁺ T cell population⁹⁻¹². Adipose T_{reg} cells have enhanced expression of genes such as Il10, Gata3, Pparg, and Ilrl1 that define their adipose and anti-inflammatory phenotype^{9,10,13,14}. Furthermore, they express high amounts of the interleukin-33 (IL-33 ) receptor ST2 (IL-1R4), and IL-33 is critical for their local expansion and transcriptional stability^{11,15}. We previously described a critical role for iNKT cell derived IL-2 to maintain T_{reg} cell numbers and to boost their function in adipose tissue³. In addition, ILC2s have been shown to play a role in T_{reg} cell homeostasis via ICOSL-ICOS interactions after IL-33 administration, and T_{reg} cells from ICOSL-deficient mice fail to expand with IL-33 treatment¹⁶. While these studies provide mechanistic insights into regulation of T_{reg} homeostasis, the basis for the marked increase in T_{reg} cell numbers with age is unknown.

IL-33 is an important factor for non-shivering thermogenesis, the metabolic adaptation to cold temperatures^{17,18}. Adaptive thermogenesis is mediated in large part by uncoupling protein 1 (UCP1), which uncouples oxidative phosphorylation from ATP synthesis to generate heat. IL-33 is critical for body temperature regulation in newborns¹⁸, and adult mice deficient in IL-33 cannot induce UCP1 and exhibit defects in thermogenesis^{8,19}. In adipose tissue, the main source of IL-33 is debated, and has been ascribed to a number of different stromal cell types including mesenchymal Cadherin-11⁺ (Cdh11) cells, podoplanin⁺ (Pdpn⁺) fibroblasts, or CD31⁺ endothelial cells^{15,16,20,21}. Importantly, the mechanisms that regulate the endogenous expression of IL-33 in adipose tissue remain elusive.

The role of γδ T cells as guardians against pathogens at barrier sites has been well documented. However, the physiological role of γδ T cells at steady state and in non-barrier tissues is less appreciated. We uncover a new biological axis where PLZF⁺ IL-17A-producing γδ T cells crosstalk with adipose stromal cells to regulate IL-33 abundance with downstream effects on T_{reg} cell accumulation and thermoregulation. Post-translational processing tightly controls the levels of IL-33³⁹, but the upstream mechanisms that control its transcription have been less studied. In addition, the main cell type that produces IL-33 in murine adipose tissue had been a matter of debate. Our studies point to Pdpn⁺ and PDGFRα⁺ as IL-17A responsive stromal cells in visceral adipose tissue. Moreover, TNF and IL-17A synergize to increase the numbers and Il33 expression, respectively, of Pdpn⁺ and PDGFRα⁺ cells to modulate IL-33 amounts in situ. These findings highlight an important homeostatic role for IL-17A in adipose tissue, independent from microbial insult.

Such effects mediated by γδ T cells are also intriguing given the previously defined roles for iNKT cells that also regulate T_{reg} cell homeostasis^{3,7}. We suggest lo that while iNKT cells play a key role in regulating T_{reg} numbers and function in young mice and via IL-2, PLZF⁺ γδ T cells play a key role in adult mice via IL-33 when iNKT cell numbers decline. Despite their important roles as regulators of type 2 immunity in young mice, ILC2s and iNKT cells decrease with age, while a new wave of immune cells composed of γδ T cells and T_{reg} cells expand. This temporal regulation of adipose lymphocytes may ensure redundancies in the molecular pathways that maintain healthy adipose tissue, which is critical for local and systemic metabolic homeostasis.

In addition, we have discovered γδ T cells and the cytokine IL-17A as critical regulators of thermogenesis, a distinctive function of adipose tissue. γδ T cell- and IL-17A-deficiency dramatically affect the ability of mice to survive upon cold challenge and robustly induce UCP1-dependent thermogenic responses. Remarkably, both iNKT cells and PLZF⁺ γδ T cells in adipose tissue regulate thermogenesis through FGF-21 and IL-17A, respectively^{3,7}. These roles of iNKT cells and γδ T cells to both regulate T_{reg} cells and thermogenesis by complementary mechanisms underscores the remarkable importance of these innate T cell populations in adipose tissue.

Methods of Treatment—Obesity

γδ T cells are canonical, meaning they have an invariant or semi-invariant T cell receptor. They produce IL-17 and TNF and express the transcription factor PLZF, unlike most γδ T cells at other sites. Their actions in adipose tissue, partly through production of synergistic IL-17 and TNF which induces IL-33 production by adipose stromal cells, enhances Tregulatory cell survival and/or expansion which is beneficial for adipose tissue and systemic health. These actions, again partly through IL-33 , also generates body heat through thermogenesis. This can induce weight loss in obesity, and can lower fasting glucose and improve metabolism and insulin resistance. We have also found that this pathway exists in human adipose tissue. The present methods can be used, e.g., in subjects who are obese (BMI of 30 kg/m² or above, calculated as weight in kilograms divided by the square of height in meters). In some embodiments, the subjects do not have diabetes, e.g., does not have type 2 diabetes.

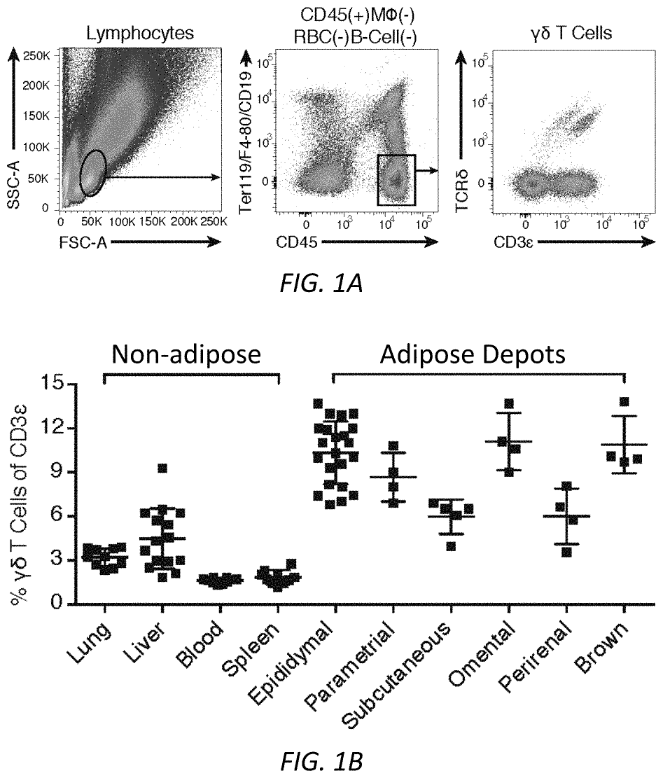

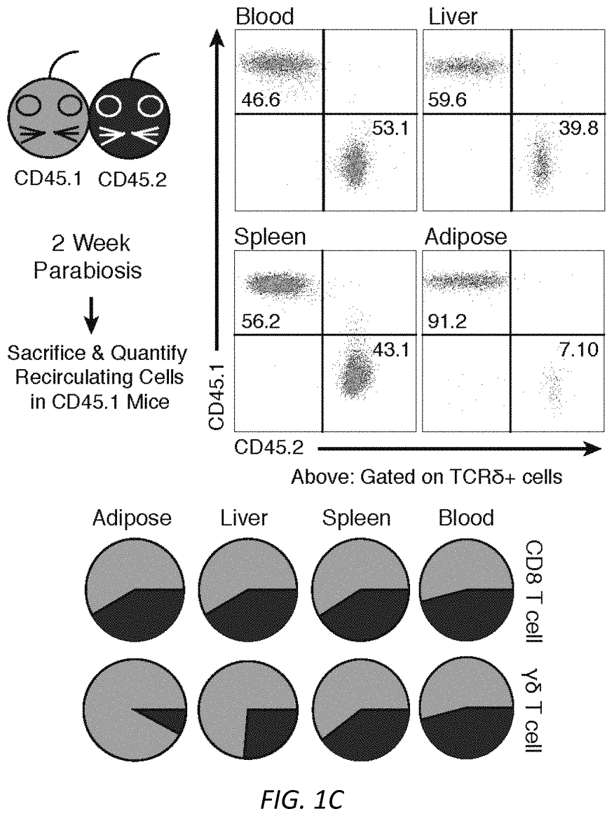

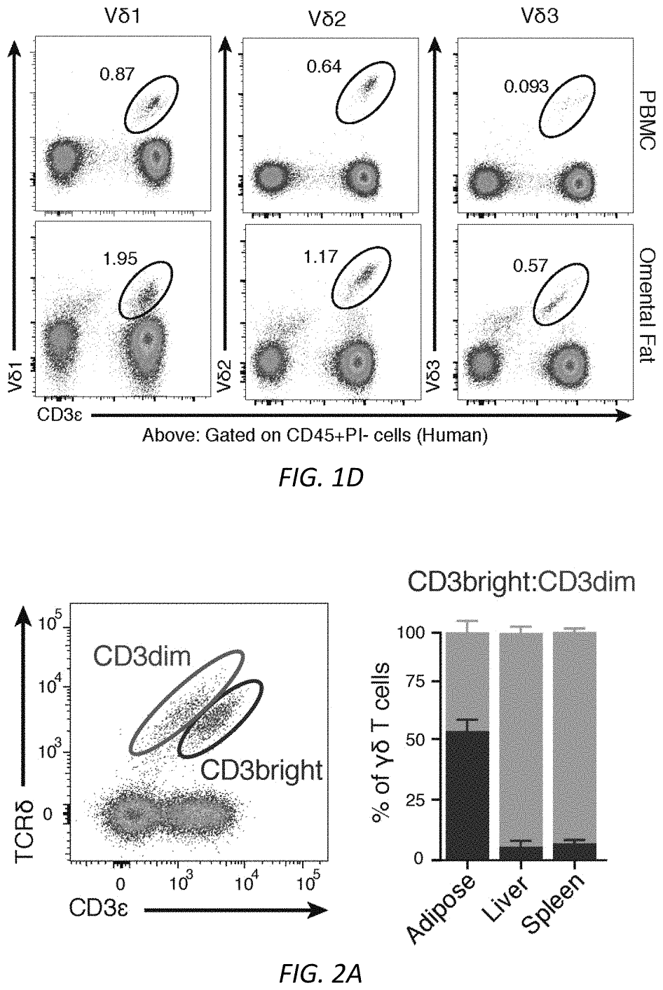

Thus, these methods can include activation of γδ T cells in adipose tissue, through stimulating their specific T cell receptor. In mice, the relevant γδ subset are Vd1Vg6. In humans, the methods can include activating Vd2Vg9 or Vg9 negative Vd2 cells; Vd1, or Vd3 cells, which are also present in human adipose tissue, may also produce IL-17 and TNFα and thus can also be activated. As shown in FIG. 1D, the Vd1,2,3 in the human adipose tissue, and FIG. 6H shows that human IL-17 and TNF induced Il-33 from human adipose stromal cells. In addition, we sequenced the human γδ population and determined that Vd2 have IL-17 associated genes (IL23R and RORA).

The γδ cells can be targeted with an activating monoclonal antibody against a specific TCR, e.g., a monoclonal antibody targeting Vd2, a number of which are available commercially, e.g., from Abcam, GeneTex, Invitrogen Antibodies, Miltenyi Biotec, and United States Biological. Activating or inhibitory antibodies can be made and identified using assays known in the art. Other methods of activating γδ T cells, can be used, e.g., administration of pyrophosphates or butyrophilins (see, e.g., Alexander et al., Clin Cancer Res. 2008 Jul. 1; 14(13): 4232-4240; Barros et al., Cell. 2016 Sep. 22; 167(1):203-218.e17; and Gu et al. PNAS Aug. 29, 2017. 114 (35) E7311-E7320 (butyrophilin 3A1 (BTN3A1)).

Alternatively or in addition, the methods can include administration of the factors produced by adipose γδ T cells that control inflammation and/or thermogenesis. These factors include, for example, a combination of TNFα (or an agonist of the TNF Receptor (TNFR)) and IL-17 (or an agonist of the IL-17 Receptor) and/or an IL-17 receptor agonist.

Administration of TNFα can include administration of the purified protein. Exemplary sequences of human TNFα are known in the art, e.g., NCBI RefSeq ID. NP_000585.2 (SEQ ID NO:1).

Human TNFα
(SEQ ID NO: 1)

| | |
|---|---|
| 1 | mstesmirdv elaeealpkk tggpqgsrrc |
| | Iflslfsfli vagattlfcl ihfgvigpqr |
| 61 | eefprdlsli splaqavrss srtpsdkpva |
| | hvvanpqaeg qlqwlnrran allangvelr |
| 121 | dnqlvvpseg lyliysqvlf kgqgcpsthv |
| | llthtis ria vsyqtkvnll saikspcqre |
| 181 | tpegaeakpw yepiylggvf qlekgdrlsa |
| | einrpdyldf aesgqvyfgi ial |

Agonists of the TNFR are also known in the art. The tumor necrosis factor receptors (TNFRs), include glucocorticoid-induced TNFR (GITR; CD357), CD27, OX40 (CD134), and 4-1BB (CD137). Agonists include Varlilumab, Urelumab, utomilumab, PRS-343, MEDI0562, MOXRO0916, GSK3174998, PF-04518600, to TRX518. See, e.g., Sturgill and Redmond, AJHO. 2017; 13(11):4-15.

Administration of IL-17 can include administration of the purified protein. Exemplary sequences of human IL-17 are known in the art, e.g., NCBI RefSeq ID. NP_002181.1 (SEQ ID NO:2), e.g., a protein comprising amino acids 69-147 of SEQ ID NO:2.

Human IL-17
(SEQ ID NO: 2)

| | |
|---|---|
| 1 | mtpgktslvs lllllsleai vkagitiprn |
| | pgcpnsedkn fprtvmvnln ihnrntntnp |
| 61 | krssdyynrs tspwnlhrne dperypsviw |
| | eakcrhlgci nadgnvdyhm nsvpiqqeil |
| 121 | vlrrepphcp nsfrlekilv svgctcvtpi |
| | vhhva |

Agonists of the IL-17R can be identified using methods known in the art.

Alternatively or in addition (e.g., in combination with TNFα), the methods can include administration of IL-33 , e.g., purified IL-33 protein. Exemplary sequences of human IL-33 are known in the art, e.g., NCBI RefSeq ID. NP_254274.1 (SEQ ID NO:3), e.g., a protein comprising amino acids 95-270, 99-270, or 109-270 of SEQ ID NO:3. Other isoforms can also be used.

Human IL-33
(SEQ ID NO: 3)

| | |
|---|---|
| 1 | mkpkmkystn kistakwknt askalcfkig |
| | ksqqkakevc pmyfmklrsg imikkeacyf |
| 61 | rrettkrpsl ktgrkhkrhl vlaacqqqst |
| | vecfafgisg vqkytralhd ssitgispit |
| 121 | eylaslstyn dqsitfaled esyeiyvedl |
| | kkdekkdkvl isyyesqhps nesgdgvdgk |
| 181 | mlmvtlsptk dfwlhannke hsvelhkcek |
| | plpdqaffvl hnmhsncvsf ecktdpgvfi |
| 241 | gvkdnhlali kvdssenlct enilfklset |

Methods of Treatment—Cachexia

We have identified a role for innate lymphocytes that are resident in adipose tissue, and the cytokines they produce, in cachexia. Cachexia is a wasting of the body (e.g., fat and muscle) due to cancers and chronic illnesses including COPD, diabetic kidney disease, heart faillure and others, and is responsible for the death of ˜30% of cancer patients. The first step in cachexia is the activation of browning in white adipose tissue, which occurs before any significant weight loss is seen in cachexic animal modes. Using parabiosis as a tool, we have shown that innate γδ T cells and iNKT are resident in adipose tissue and produce cytokines including TNF and IL-17 in response to changes in the environment including cold exposure and changes in the diet. In addition to cytokines usually associated with these immune cells, transcriptomics on these subsets in adipose tissue has revealed that these cells also produce other factors that can modulate neurons, which are also involved in thermogenesis. The activation of these innate resident T cells induced browning of white adipose tissue and thermogenesis and increased systemic energy expenditure, which are the key steps in induction of cachexia.

Thus, these methods can include blocking the activation of γδ T cells and/or iNKT cells in adipose tissue, or depletion of these cells (e.g., of Vd2Vg9 or Vg9 negative Vd2 γδ T cells; Vd1 γδ T cells; or Vd3 γδ T cells) through selective depleting antibodies against their specific T cell receptor or surface markers. Antibodies specific for the iNKT cell TCR for activation are known in the art, e.g., the 6b11 antibody (Exley et al., Eur J Immunol. 2008 June; 38(6): 1756-1766) targets the iNKT TCR).

For example, in some embodiments the methods can include blocking the factors produced by adipose iNKT, γδ T cells and ILC3, that control inflammation and/or thermogenesis could be new treatments for cachexia or other wasting diseases. For example, the methods can include administration of an inhibitor of IL-17 or the IL-17R, e.g., Secukinumab, ustekinumab, brodalumab or ixekizumab; see, e.g., Rizvi et al., Nature Reviews Drug Discovery 14,745-746 (2015). These methods can also include administration of an inhibitor of TNFα or the TNFR, e.g., a monoclonal antibody such as infliximab, adalimumab, certolizumab pegol, and golimumab, or with a circulating receptor fusion protein such as etanercept. The inhibitors of IL-17/IL-17R (e.g., Brodalumab and others) and of TNFα/TNFR can be administered together, e.g., in a single composition, or in separate compositions.

Alternatively or in addition, the methods can include administering an IL-33 antibody, e.g., a long-acting IL-33 neutralizing antibody. The sequence of human IL-33 is known in the art (see above). Anti-IL-33 antibodies are known in the art; see, e.g., WO2014164959A2, and are commercially available from Abbexa Ltd; Abcam; ABclonal; Abnova Corporation; antibodies-online; Assay Biotech; AssayPro; Atlas Antibodies; Aviva Systems Biology; BioLegend; Biomatik; Bio-Rad; Biorbyt; Bioss Inc.; BioVision; BosterBio; Cell Sciences; Cloud-Clone; Creative Biolabs; Creative Diagnostics; Elabscience Biotechnology Inc.; Enzo Life Sciences, Inc.; Fitzgerald Industries International; GeneTex; IBL America Immuno-Biological Laboratories); Invitrogen Antibodies; LifeSpan BioSciences; MBL International; MilliporeSigma; MyBioSource.com; Novus Biologicals; NSJ Bioreagents; OriGene Technologies; PeproTech; ProSci, Inc; Proteintech Group Inc; R&D Systems; Raybiotech, Inc.; Rockland Immunochemicals, Inc.; Santa Cruz Biotechnology, Inc.; Signalway Antibody LLC; Sino Biological; SouthernBiotech; St Johns Laboratory; United States Biological; and Abbexa Ltd.

Pharmaceutical Compositions and Methods of Administration lo The methods described herein include the use of pharmaceutical compositions comprising one or more active ingredients as described herein. In some embodiments, no other active ingredients are used or administered.

Pharmaceutical compositions typically include a pharmaceutically acceptable carrier. As used herein the language “pharmaceutically acceptable carrier”includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of lo manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated herein. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a lo flavoring agent such as peppermint, methyl salicylate, or orange flavoring.

In some embodiments, the proteins or antibodies can be modified, e.g. pegylated to extend half-life. Methods for producing recombinant protein and/or purifying native proteins are known in the art. In some embodiments, the methods include administration by injection into adipose tissue, to target adipose-resident cells. In some embodiments, the dug is formulated to target adipose, e.g., using adipo-8 aptamers (Liu et al., May 25, 2012; doi.org/10.1371/journal.pone.0037789). In some embodiments, an extended release formulation is used.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. Nanoparticles (1 to 1,000 nm) and microparticles (1 to 1,000 pm), e.g., nanospheres and microspheres and nanocapsules and microcapsules, can also be used, e.g., adipose-targeted nanoparticles (see, e.g., Xue et al., PNAS May 17, 2016. 113 (20) 5552-5557).

The pharmaceutical compositions can be included in a container, pack, or dispenser together with instructions for use in a method described herein.

### EXAMPLES The invention is further described in the following examples, which do not limit the scope of the invention described in the claims.

Methods

The following materials and methods were used in the Examples below.

Mice. C57BL/6J (WT), Il17a^{-/-}, Tcrd^{-/-}, and Il17a^{GFP} mice were purchased from Jackson Laboratory. Littermates were bred and maintained in specific-pathogen-free animal facilities at Brigham and Womens Center for Comparative Medicine. In almost all experiments, male mice of specified age were used. PLZF to reporter mice (Zbtb16^{GFP} mice) and Zbtb16^{-/-} were generated in the SantAngelo laboratory as previously described^{3,40}. Vgamma4/6^{-/-} (Vg4/6^{-/-}) mice were a kind gift from R. O'Brien³⁰. All studies were executed by following relevant ethical regulations detailed in animal use protocols. All animal work and protocols were approved by and was in compliance with the Institutional Animal Care and Use Committee guidelines of Brigham and Womens Hospital and Harvard Medical School.

Parabiosis. Parabiosis studies were conducted as previously described⁴¹. CD45.1⁺ and CD45.2⁺ mice were first anesthetized with ketamine (100 mg per kg body weight) and xylazine (10 mg per kg body weight). After mice were shaved, a linear incision was made from the scapulae to the lower abdomen on opposing sides of each member of the pair. Mice were placed side by side and skin edges were sewn together. Each pair was housed singly, with food placed on the floor of the cage for the first week during recovery. Parabiotic mice were kept together for 2-3 weeks. Chimerism in the blood and tissues was defined for gated lymphocytes, or lymphocyte subsets as the percentage of CD45.1⁺ cells over the percentage of CD45.1⁺ cells plus CD45.2⁺ cells in CD45.2⁺ mice, and as the percentage of CD45.2⁺ cells over the percentage of CD45.2⁺ cells plus CD45.1⁺ cells in CD45.1⁺ mice.

CLAMS. Indirect calorimetry experiments were performed with a Comprehensive Lab Animal Monitoring System (CLAMS, Columbus Instruments) essentially as described⁴². Mice were surgically implanted with intraperitoneal wireless temperature transmitters. Following recovery, mice were housed in the CLAMS and maintained at thermoneutrality for 3 days. To observe the response to adrenergic agonist versus effects of injection alone, mice housed at thermoneutrality were first injected with a control solution of sterile saline (200 μl) and monitored for 3 h. The animals were then injected (1 mg/kg) with the selective β3-adrenergic receptor agonist CL316,243 (Sigma Chemical Co.) and monitored as indicated. For 4° C. cold challenge, mice were gradually shifted from 30° C. to 4° C. at a continuous rate over 3 h. For each experimental condition, metabolic variables were adjusted for differences in body composition by analysis of covariance (ANCOVA) in the R programming language with a custom package for indirect calorimetry experiments (CalR).

Tissue digestion. Adipose tissue was carefully excised, minced, and digested with 1 mg/ml collagenase type 2 (Worthington #LS004188) in RPMI (Life Technologies) for 25 min at 37° C., with shaking. Cells were passed through a 70 μm cell strainer, washed, and centrifuged for 5 min at 300×g to pellet the stromal-vascular fraction from floating mature adipocytes. For the preparation of single-cell suspensions of liver, the organ was perfused via the portal vein with 10 ml PBS and mashed through a 70 μm cell strainer. Liver samples underwent enrichment for lymphocytes by centrifugation using a Percoll gradient. Spleens were strained through a 70 μm cell strainer and spun. Pellets from all tissues were subjected to red blood cell lysis and subsequently resuspended in flow cytometry buffer (2% FBS and 0.02% NaN₃ in phenol-free DMEM) for further staining.

Flow cytometry and cell sorting. Single-cell suspensions were incubated with Fc receptorblocking antibody (14-0161-82; eBioscience) before being stained on ice. Dead cells were excluded with a live/dead Zombie Aqua stain (BioLegend). For intracellular transcription factor staining, cells were fixed and permeabilized using the eBioscience Transcription Factor Fix/Perm Buffer. Mouse antibodies were as follows: anti-CD45 (30-F11), anti-CD45.1 (A20), anti-CD45.2 (104), anti-CD27 (LG.3A10), anti-CD69 (FN50), anti-CD44 (BJ18), anti-CD127 (A019D5), anti-CD4 (RM4-5), anti-CD8□ (53-6.7), anti-TCRP (H57-597), anti-KLRG1 (2F1/KLRG1), anti-pdpn (8.1.1), anti-CD26 (H194-112FC), and anti-CD31 (390) were all purchased from BioLegend. Anti-Ter119 (TER-119), anti-F4/80 (BM8), anti-CD19 (ID3), anti-γδ TCR (GL3), anti-CD3e (500A2), anti-PLZF (Mags.21F7), anti-Foxp3 (FJK-16s), anti-ST2 (RMST2-2), anti-T-bet (4B10), anti-RORγt (B2D), anti-TNF (MP6-XT22), anti-IL-17A (17B7), anti-IFN-γ (XMB1.2), anti-PDGFRα (APA5), anti-IL-17RA (PAJ-17R), and streptavidin-APC were all purchased from eBioscience. To stain for Vγ6⁺ cells, GL3 (anti-TCRγδ) and 17D1 (anti-Vγ5Vδ1) antibodies were used as previously described⁺. Anti-Vγ4 (UC3-10A6) was purchased from BD Pharmingen. Biotinylated anti-cad11 was generated in house. Mouse PBS-57-loaded CD1d tetramers were from the NIH tetramer facility. Human antibodies were as follows: anti-TCR Vδ1 (REA173) and anti-TCR Vδ2 (123R3) were purchased from Miltenyi Biotec. Anti-CD3 (UCHT1) was purchased from BioLegend. Anti-TCR Vδ3 was custom made in Labpan (Europe). After staining, cells were passed through a 70 μm filter, and data acquired on a BD FACSAria Fusion, BD Fortessa, or BD Canto II analyzer using FACSDiva software. Spherotech AccuCount Fluorescent particles were added for cell quantification prior to analysis on the flow cytometers. Cell doublets were excluded by comparison of side-scatter width to forward-scatter area. For analysis of γδ T cells, a ‘dump gate’with Ter119, CD19, and F4/80 was used for elimination of nonspecific staining.

PLZF⁺ and PLZF⁻ γδ T cells were sorted directly from freshly digested adipose tissue using PLZF^{GFP} mice. Fibroblast subsets were sorted according to the gating scheme outlined in FIG. 6d. Cell sorting was performed on a BD FACSAria Fusion sorter using a 70 μm nozzle. Cell purity was routinely >98%. For RNA analyses, sorted cells were lysed in either Trizol (Qiagen) or RLT lysis buffer (Qiagen) with 1% β-mercaptoethanol (2-ME, Sigma).

Immunofluorescence. For detection of GFP, epididymal adipose tissue was harvested into 0.02% sodium azide and 5% normal mouse serum in PBS (Jackson ImmunoResearch Laboratories). Adipose tissue mounted on glass slides with Aqua Poly/Mount (Polysciences) and a coverslip was imaged by confocal microscopy (Leica TCS SP5).

In vitro stimulations. Adipose tissue was digested as described above and bulk stromal vascular fraction (SVF) was stimulated with phorbol 12-myristate 13-acetate (PMA, 50 ng/ml) and ionomycin (1 μg/ml) for 6 h in complete RPMI medium (RPMI supplemented with 10% FBS [Gemini], HEPES [Invitrogen], L-glutamine, penicillin/streptomycin, and 2-ME). Brefeldin A (1:1000, eBioscience) was added for the last 5 h. Cells were washed twice in 2% FBS in DMEM, surface stained, and fixed and permeabilized using the eBioscience Transcription Factor Fix/Perm Buffer to assay cytokine production by γδ T cells.

Primary stromal cells from mouse and human adipose tissue were generated by digesting adipose tissue and expanding bulk cells in 6-well plates in Dulbeccos modified Eagles medium (DMEM) supplemented with 10% fetal bovine serum (FBS; Gemini), 2 mM L-glutamine, 50 μM 2-ME, and antibiotics (penicillin and streptomycin). After 3-5 days, non-adherent cells are washed off and stromal cells trypsinized. Stromal cells were plated on day 1 at 5×10⁴ cells per well in 24-well plates in 10% FBS-containing media. Human cells were serum starved on day 2 by changing to 1% FBS-containing media. Cells were left unstimulated or stimulated with the indicated concentrations of TNF (Peprotech), IL-17A (Peprotech), or a combination of the two, for 18 h prior to washing in PBS and harvesting cell lysates for protein analysis.

Immunoblotting. Whole adipose tissues were homogenized in lysis buffer (50 mM Tris-HCl, 150 mM NaCl, 5 mM EDTA, 1% Triton X-100, Protease inhibitor cocktail (Roche), 500 U/ml benzonase nuclease (Novagen), 1 mM PMSF, 1 mM Na₃VO₄, 10 mM NaF). Samples were clarified by centrifugation for 15 min at 16,100×g. Protein concentration was measured with a BCA protein assay kit (Pierce). 20-50 μg of protein was loaded on 4-20% Mini Proteon TGX gradient gels (Bio-Rad). Protein was transferred to 0.2 μm PVDF membranes (Bio-Rad). Membranes were blocked in Tris-buffered saline plus 0.1% Tween 20 (TBS-T) containing 5% BSA or 5% milk for 1 h at 25° C. followed by overnight incubation with primary antibody at 4 °C. Primary antibodies were diluted 1:1000 in 5% BSA or 5% milk in TBS-T. Primary antibodies used were against UCP1 (Abcam, #10983) and HSP90 (Cell signaling, #4877, C45G5). Membranes were washed with TBS-T and incubated with HRP-conjugated secondary antibodies for 1 h at 25° C. HRP was activated with Bio-Rad Clarity Western ECL Substrate (Bio-Rad) and visualized with a chemiluminscent detection system using Bio-Rad Chemidoc. Densitometry of blots were analyzed using ImageJ.

ELISA. Processed adipose SVF lysates or stromal cell cultures were diluted 1:2 in reagent diluent (1% BSA in PBS) and IL-33 protein concentrations quantified using Mouse/Rat IL-33 Quantikine ELISA kit (M3300, R&D Systems). Adipose SVF lysates were similarly analyzed for IL-2 using Ready-SET-Go!® ELISA kit (eBioscience).

RT-PCR analyses. Tissues were snap frozen in liquid nitrogen and stored at −80°C. until use. Inguinal, epididymal, and brown adipose tissue depots were homogenized in TRIzoI® Reagent (#15596026, Life Technologies) and mixed with chloroform at a ratio of 5:1. After spinning, the upper aqueous phase was mixed with the same volume of 70% EtOH and RNA was isolated using RNeasy Mini Kits (#74104, Qiagen). cDNA was prepared using Quantitect RT-PCR (#205311 Qiagen) and PCR performed with Brilliant III SYBRGreen (#600882, Agilent Technologies) on a Stratagene Mx3000. Primers used were as follows: Tbp (forward: 5′-CTACCGTGAATCTTGGCTGTAAAC-3′(SEQ ID NO:1); reverse: 5′-AATCAACGCAGTTGTCCGTGGC-3′(SEQ ID NO:2)), Il10 (forward: 5′-AATAAGCTCCAAGACCAAGG-3′(SEQ ID NO:3); reverse: 5′-CAGACTCAATACACACTG-3′(SEQ ID NO:4)), Il33 (forward: 5′-ATGGGAAGAAGCTGATGGTG-3′(SEQ ID NO:5); reverse: 5′-CCGAGGACTTTTTGTGAAGG-3′(SEQ ID NO:6)), Ucp1 (forward: 5′-GGCCTCTACGACTCAGTCCA-3′(SEQ ID NO:7); reverse: 5′-TAAGCCGGCTGAGATCTTGT-3′(SEQ ID NO:8)), Ppargc1a (forward: 5′-AGCCGTGACCACTGACAACGAG-3′(SEQ ID NO:9); reverse: 5′-GCTGCATGGTTCTGAGTGCTAAG-3′(SEQ ID NO:10)), Dio2 (forward: 5′-TGCCACCTTCTTGACTTTGC-3′(SEQ ID NO:11); reverse: 5′-GGTTCCGGTGCTTCTTAACC-3′(SEQ ID NO:12)), Cox7a1 (forward: 5′-AAACCGTGTGGCAGAGAAGCAG-3′(SEQ ID NO:13); reverse: 5′-CCCAAGCAGTATAAGCAGTAGGC-3′(SEQ ID NO:14)), Adrb3 (forward: 5′-AACTGAAACAGCAGACAGGGAC-3′(SEQ ID NO:15); reverse: 5′-CCCCCATGTACACCCTAGTT-3′(SEQ ID NO:16)), Th (forward: 5′-CCAAGGTTCATTGGACGGC-3′(SEQ ID NO:17); reverse: 5′-CTCTCCTCGAATACCACAGC-3′(SEQ ID NO:18), Lipe (forward: 5′-GCTCATCTCCTATGACCTACGG-3′(SEQ ID NO:19); reverse: 5′-TCCGTGGATGTGAACAACCAGG-3′(SEQ ID NO:20)), and Pnpla2 (forward: 5′-GGAACCAAAGGACCTGATGACC-3′(SEQ ID NO:21); reverse: 5′-ACATCAGGCAGCCACTCCAACA-3′(SEQ ID NO:22)).

Histology. For whole fat tissue staining, 5 mm² of brown adipose tissue was fixed in 4% of paraformaldehyde in PBS overnight, washed in PBS, and stored in 70% ethanol. Samples were processed and embedded in paraffin and stained with H&E by the Dana Farber Rodent Histopathology Core. For lipid droplet quantification, histological sections of brown adipose tissue were placed under a microscope, and images were acquired under 100× agnification. These images in TIFF format were analyzed using the automated Fiji-based Open Source software package Adiposoft. Under 0.485 microns per pixel, a minimum diameter of 20 μm and a maximum diameter of 100 μm was set for the calculation of adipocyte area.

RNA sequencing. RNA was isolated from 800-1,000 cells from sorted γδ T cell populations using Zbtb16^{GFP} mice as described. 5 μl of total RNA were placed in wells of a 96-well plate and RNA sequencing libraries were prepared at Broad Technology Labs at the Broad Institute of Harvard and MIT using the Illumina SmartSeq2 platform. Samples were sequenced on a NextSeq500 using 75 bp paired-end reads to an average depth of 9M pairs of reads per sample by the Broad Genomics to Platform. Reads were mapped to the mouse genome (mm10) using HISAT⁴³ (0.1.6-beta release). Bam files were sorted and indexed by SAMtools⁴⁴ (1.2 release). Assembly, quantification and normalization were performed using CuffLinks⁴⁵ (1.2 release), according to the Tuxedo pipeline⁴⁶. A merged transcriptome constructed from all samples was used as a reference annotation for quantification (by CuffQuant) and normalization (by CuffNorm) stages. Differentially expressed genes between PLZF⁺ and PLZF⁻ γδ T cell subsets were identified using CuffDiff⁴⁷ (false-discovery rate and adjusted P-value<0.1). Genes with calculated FPKM values (according to CuffDiff's pooled dispersion measure) lower than 2⁶ in both subsets were removed from the analysis to avoid low noisy measurements. For heatmaps, values lower than one were replaced by 1, and then data was loge transformed.

Human tissue. Omental adipose tissue was obtained from patients undergoing weight-loss surgery with approval of the Brigham and Womens Hospital Institutional Review Board. Tissue was processed similar to mouse adipose tissue. Matched peripheral blood was also collected for analysis. Informed consent was obtained from all patients and samples collected following BWH ethical regulations. Cultured stromal cell fibroblast lines were generated from visceral preadipocytes (Lonza, Cat#PT-5005) and subcutaneous preadipocytes (ATCC, Cat#PLS-210-010) and grown in T-75 cm² flasks in Dulbeccos modified Eagles medium (DMEM) supplemented with 10% fetal bovine serum (FBS; Gemini), 2 mM L-glutamine, 50 μM 2-ME, and antibiotics (penicillin and streptomycin).

Statistics. Independent experiments were repeated at least two to three times, and the data were presented as mean±SEM. Statistical significance was determined using the Students t test, one-way ANOVA, or two-way ANOVA as indicated. P-value of <0.05 was considered to be statistically significant and is presented as * P<0.05, ** P<0.01, *** P<0.001, or **** P<0.0001. No exclusion of data points or mice was used. Pilot studies were used for estimation of the sample size required to ensure adequate power. GraphPad Prism 6 was used for all statistical analyses.

Life Sciences Reporting Summary. Further information on experimental design and reagents is available in the Life Sciences Reporting Summary.

Data availability. RNAseq expression data has been deposited in the gene expression omnibus under Series GSE103742.The data that support the findings of this study are additionally available from the corresponding author upon request.

### Example 1. γδ T Cells are Enriched and Resident in Adipose Tissue

We profiled visceral adipose tissue using flow cytometry and found γδ T cells to be highly enriched in adipose tissue compared to other organs (FIGS. 1A,B). γδ T cells were abundant across several adipose depots compared to peripheral sites including lung, liver, blood, and spleen (FIG. 1B). To determine if the enriched population of γδ T cells in visceral adipose tissue was constantly replenished from the periphery or if they represented a tissue-resident population, parabiosis experiments were performed using congenic CD45.1 and CD45.2 mice (FIG. 1C). While CD8⁺ T cells reached near-complete chimerism for blood, spleen, liver, and adipose, γδ T cells showed full recirculation only in the blood and spleen, while the liver and more strikingly, adipose tissue, showed reduced chimerism (FIG. 1C). In adipose tissue, >90% of γδ T cells were endogenous to the host mice, supporting a resident or long-dwelling phenotype. Importantly, γδ T cells, identified by their Vδ chain usage, were also found in human omental adipose tissue (FIG. 1D). When compared to peripheral blood, Vδ1, Vδ2, and Vδ3 subsets were increased in human omentum as a percentage of CD45⁺ lymphocytes. Thus, γδ T cells are enriched and resident in murine and human adipose tissues.

### Example 2. PLZF Discriminates two γδ T Cell Populations

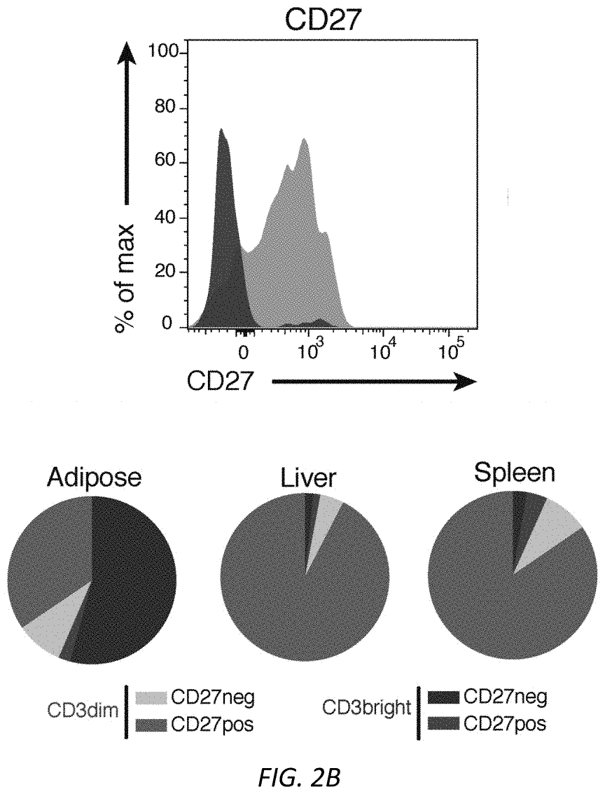

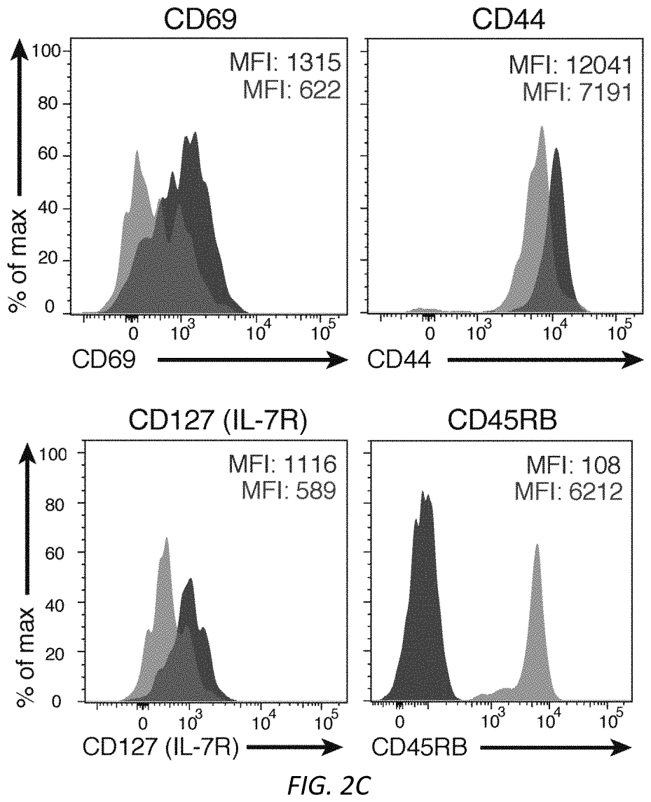

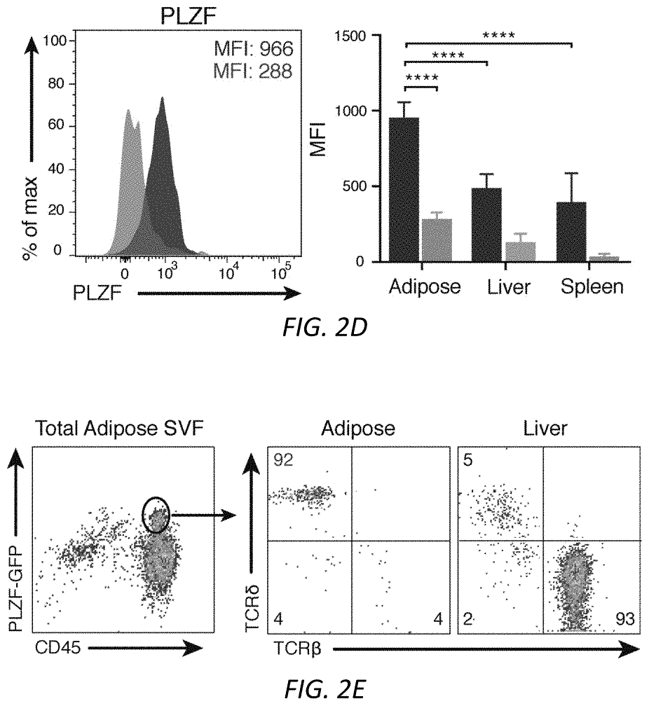

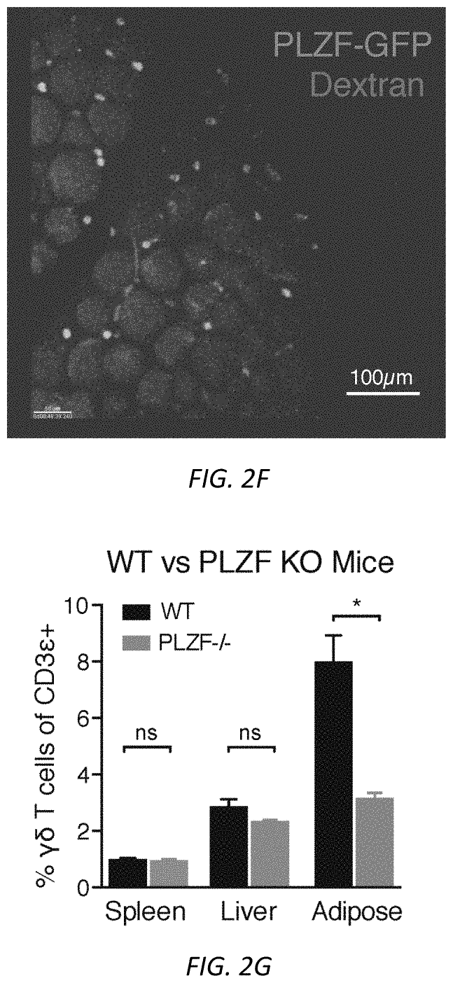

When profiling γδ T cells in adipose tissue, we found they could be separated into two distinct populations based on CD3_{ε} intensity (FIG. 2A)²². CD3_{ε}^{hi} γδ T cells were more abundant compared to CD3_{ε}^{lo} cells in adipose tissue, making up to two-thirds of the total γδ T cell pool, while other organs such as the liver and spleen had few CD3_{ε}^{hi} cells (FIG. 2A). CD27, a TNFR superfamily member and costimulatory molecule, demarcates functionally distinct γδ subsets in mice²³. We found that adipose γδ T cells were largely CD27⁻, which corresponded to the CD3_{ε}^{hi} subset, whereas in the spleen and liver, γδ T cells were largely CD3_{ε}^{lo}CD27⁺ (FIG. 2B). Additional phenotyping showed the enriched population of CD3_{ε}^{hi}CD27⁻ γδ T cells to be CD69^{hi}CD44^{hi}CD127⁺CD45RB⁻ (FIG. 2C).

Promyelocytic leukemia zinc finger protein (PLZF) is a BTB-POZ transcription factor encoded by Zbtb16 and imparts lymphocytes with innate-like qualities. PLZF is expressed on certain γδ T cells from other organs^{24,25}, iNKT cells²⁶ and human MAIT cells²⁷. We analyzed PLZF expression in adipose γδ T cells by flow cytometry and found that CD3_{ε}^{hi}γδ T cells highly expressed PLZF compared to CD3_{ε}^{lo} cells by flow cytometry (FIG. 2D). PLZF expression was significantly higher in CD3_{ε}^{hi} γδ T cells in adipose tissue compared to those in liver and spleen (FIG. 2D). Furthermore, using Zbtb16^{GFP} mice, we found that almost all the PLZF signal (>92% of PLZF⁺ cells) from adipose CD45⁺ cells was attributed to γδ T cells, while in the liver most of the PLZF⁺CD45⁺ lymphocytes were TCRβ⁺ cells (FIG. 2E). Whole mount staining of adipose tissue from Zbtb16^{GFP} mice further confirmed the presence of PLZF⁺ cells and found them to be interspersed between adipocytes (FIG. 2F). PLZF-deficient (Luxoid) mice showed a two-thirds reduction in the frequency of adipose γδ T cells, corresponding with the relative frequency of PLZF⁺CD3_{ε}^{hi} γδ T cells in adipose tissue (FIG. 2G). γδ□T cell frequencies in other organs were unaffected by loss of PLZF, as they harbored fewer PLZF⁺CD3_{ε}^{hi} γδ T cells (FIG. 2G), highlighting the requirement of PLZF for the CD27⁻ γδ T cell population resident in adipose tissue.

### Example 3. γδ T Cells are Important for Adipose T_{reg} Accumulation

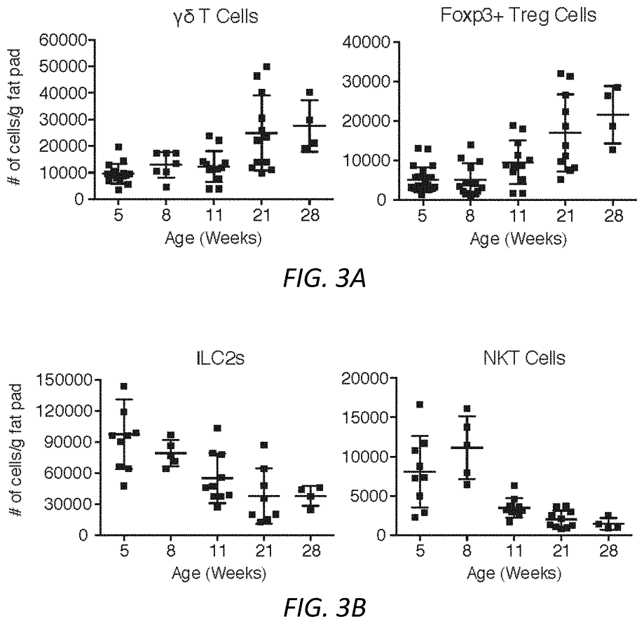

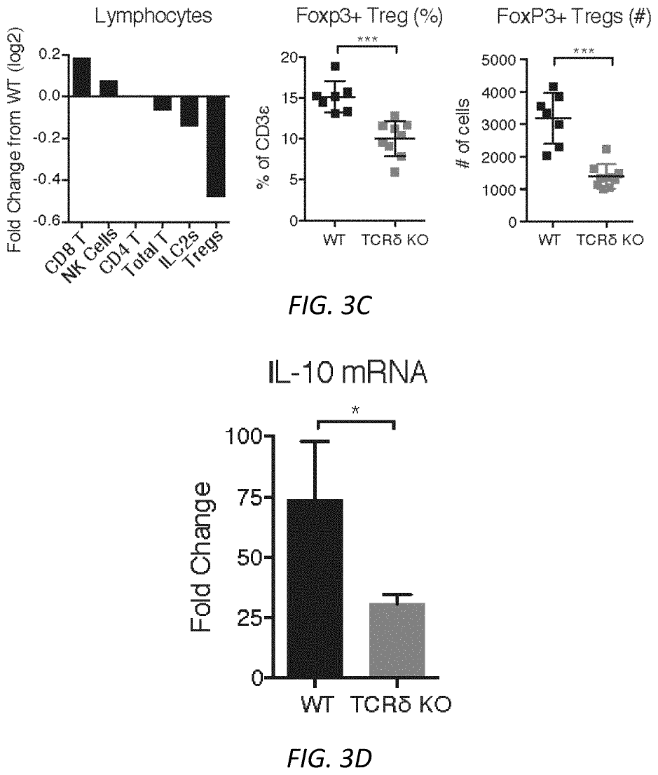

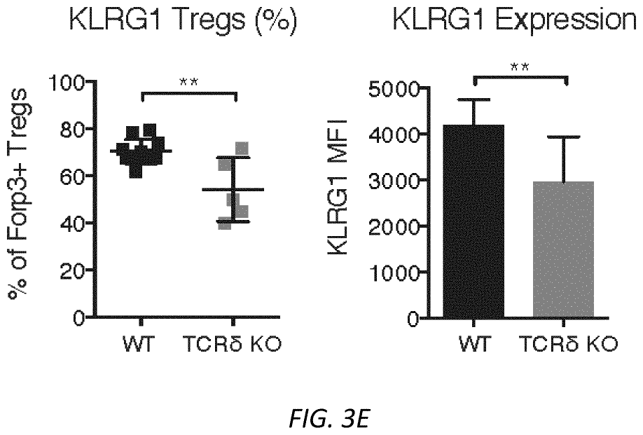

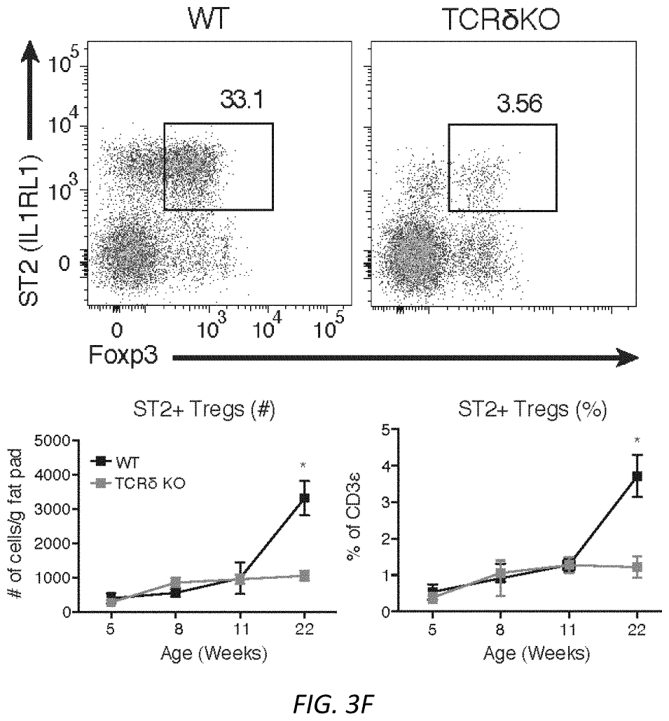

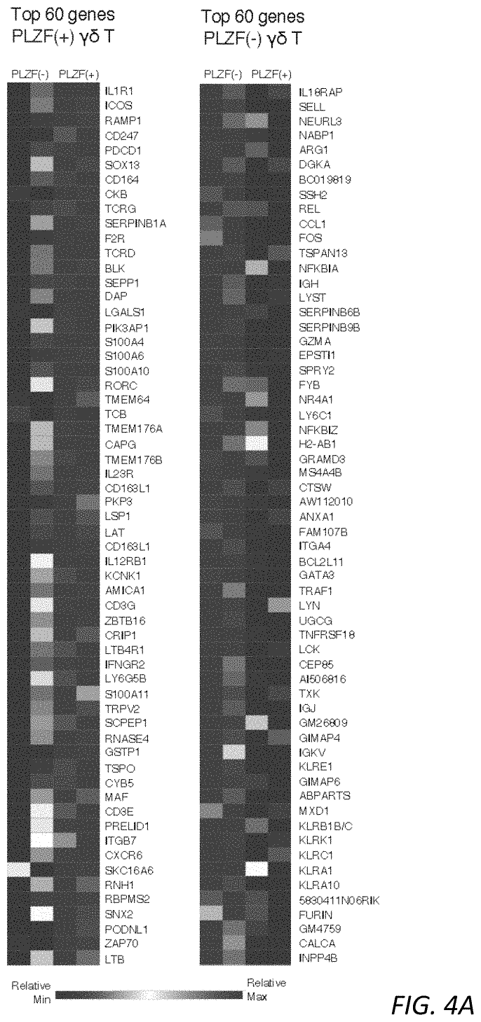

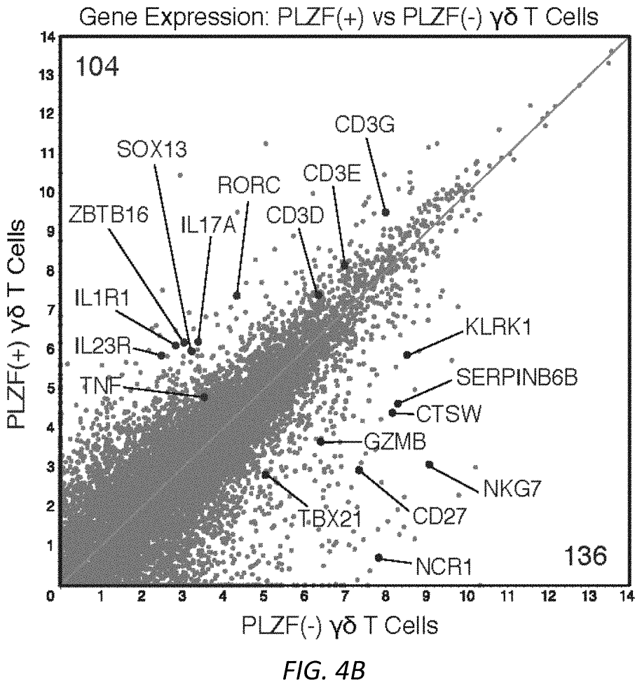

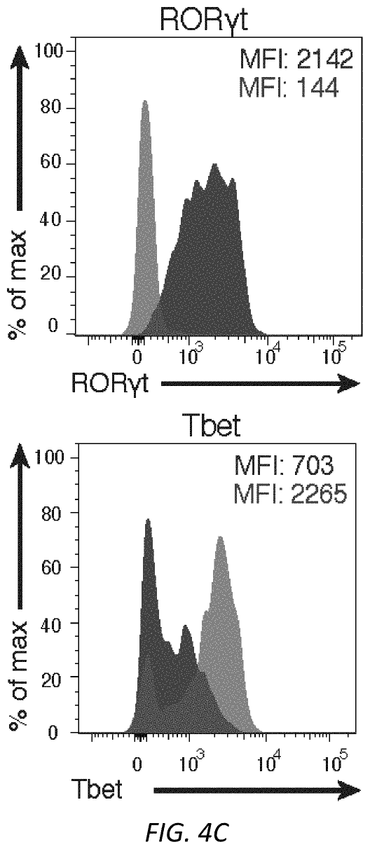

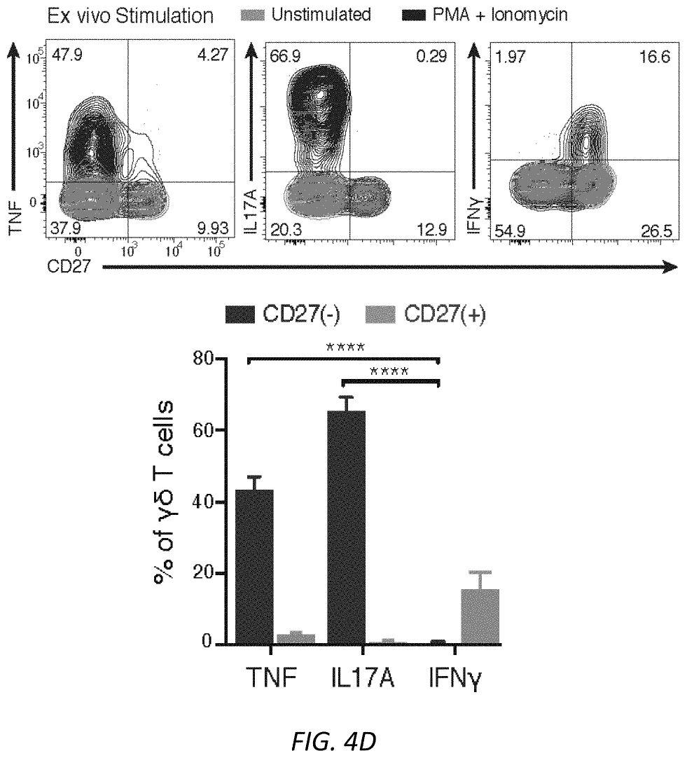

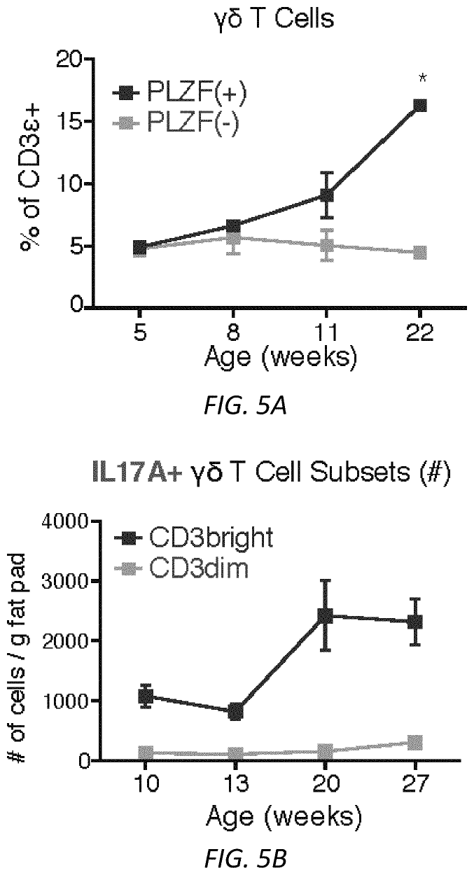

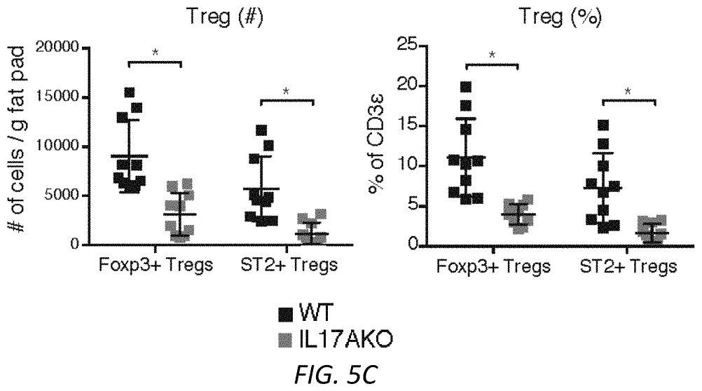

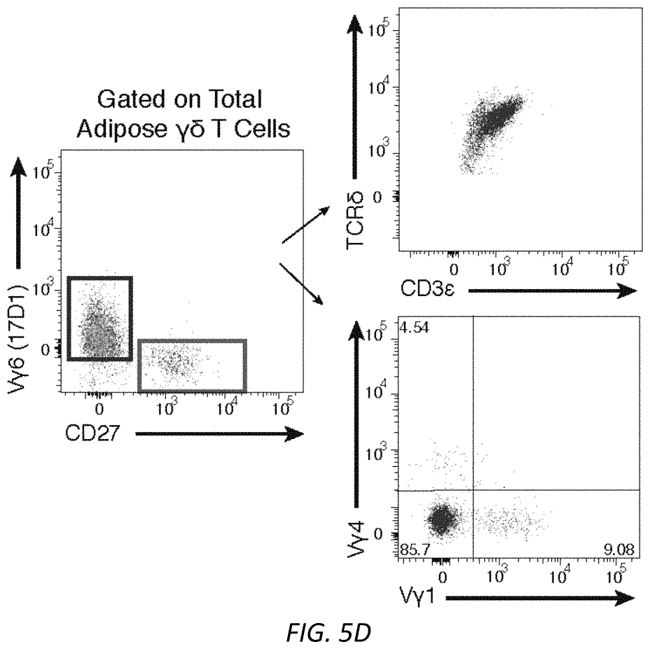

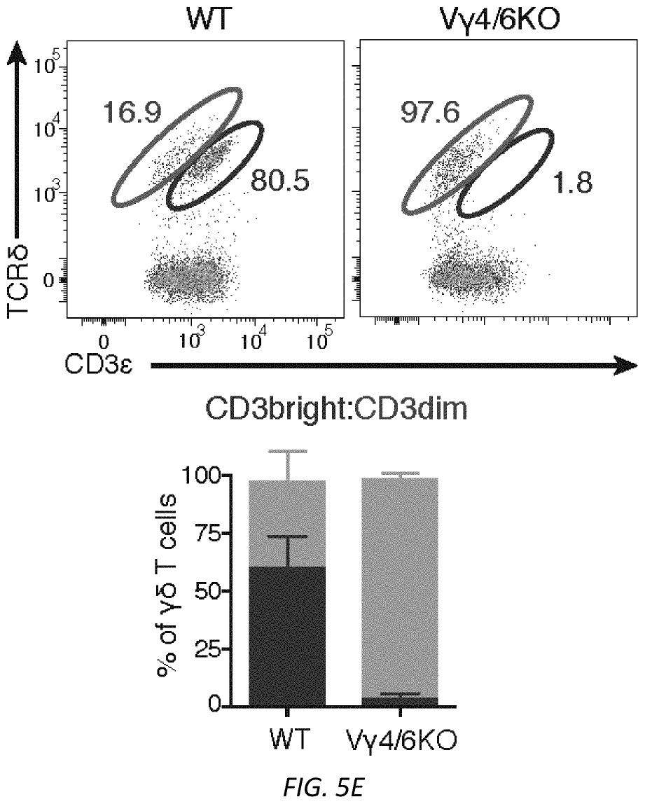

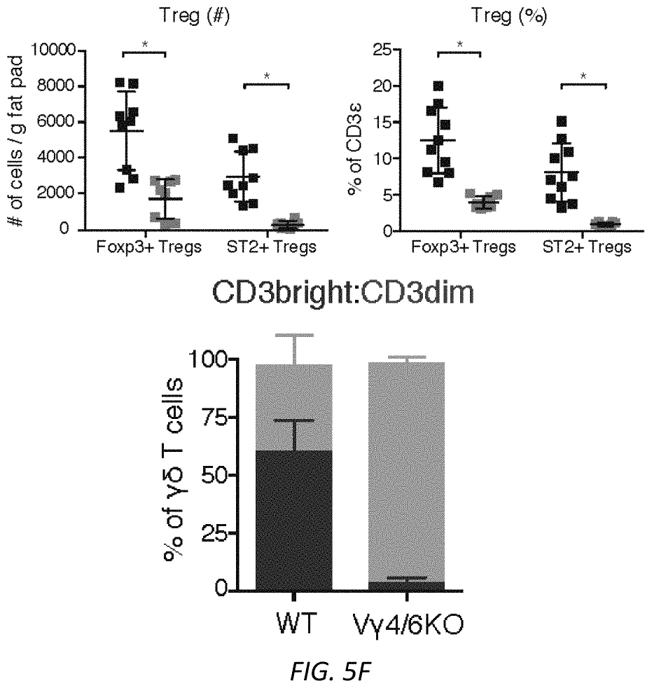

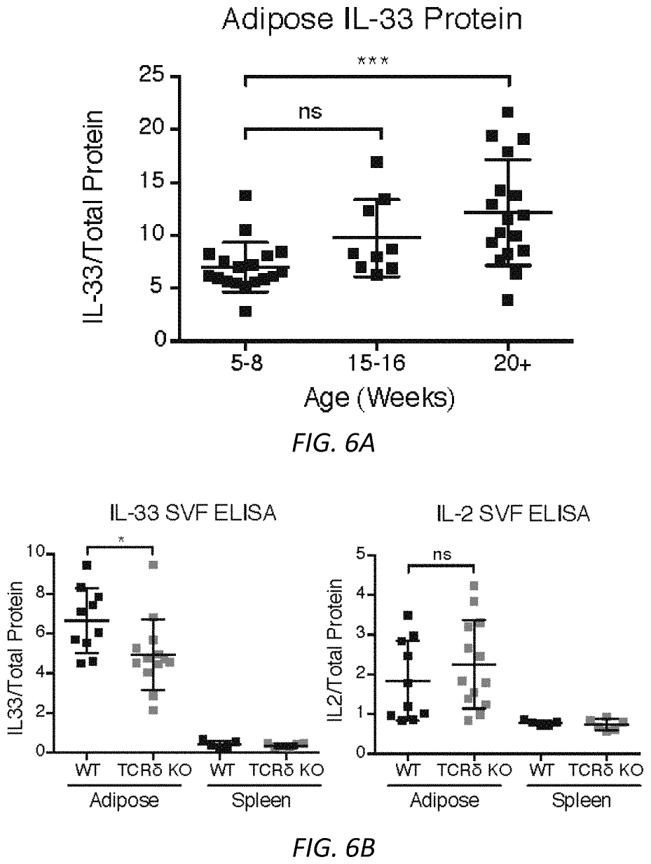

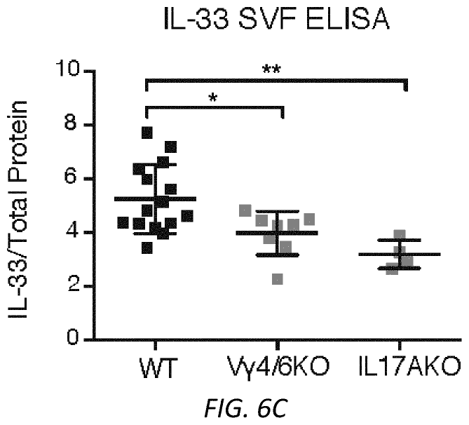

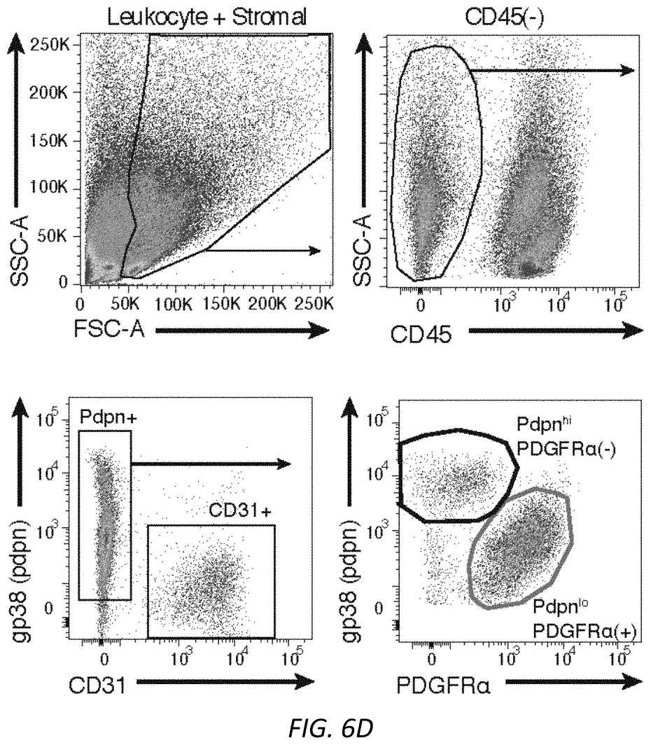

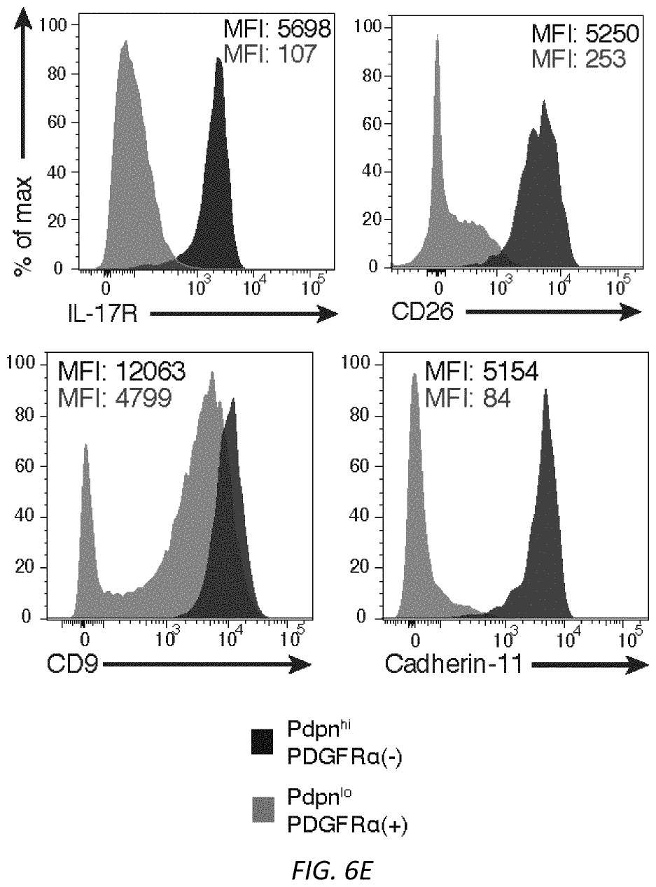

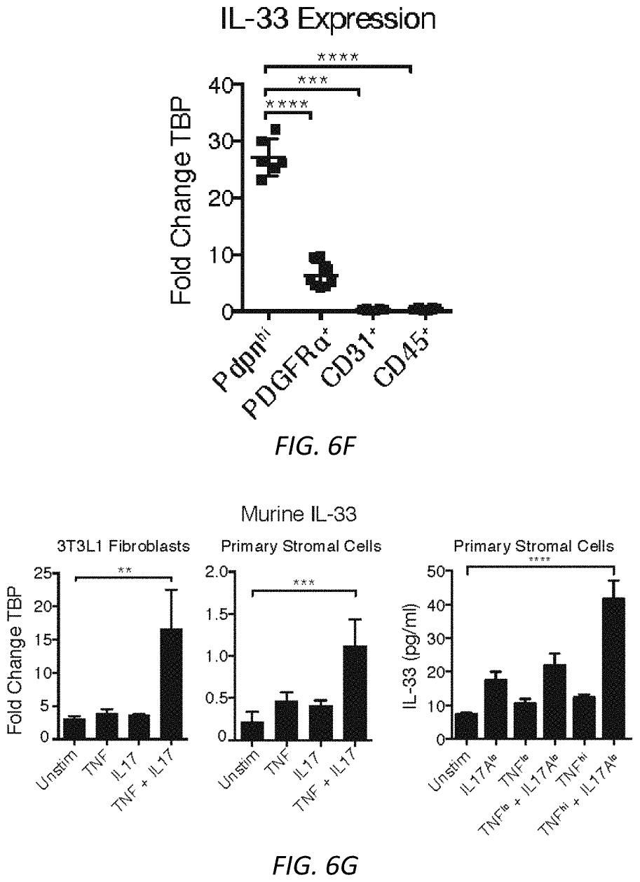

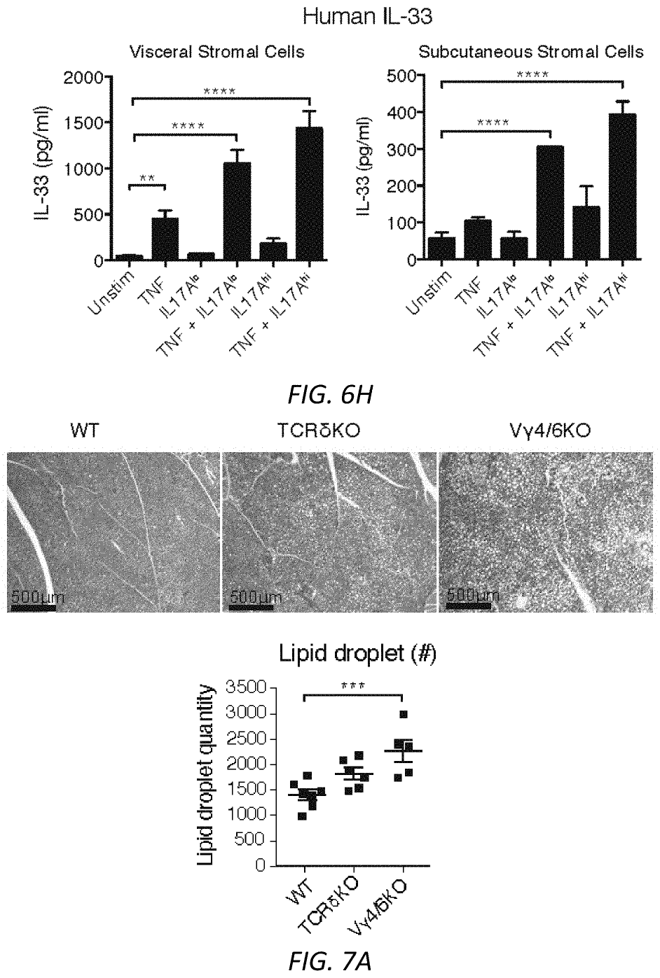

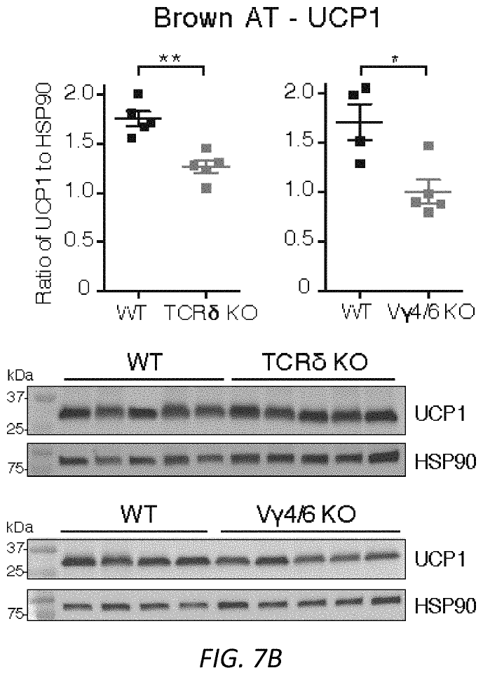

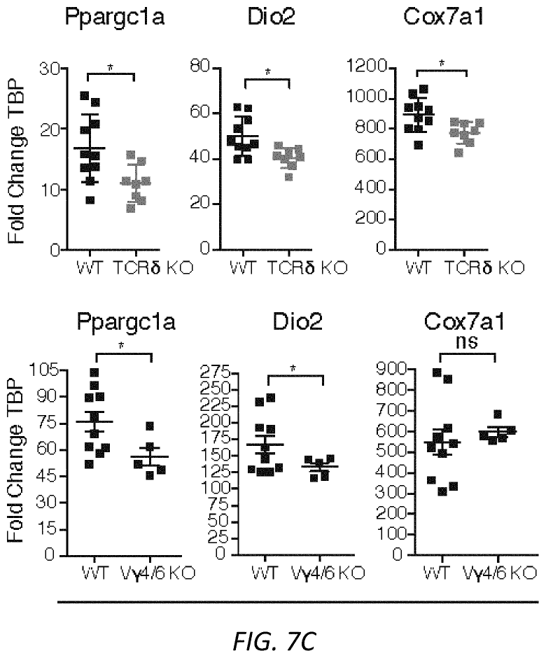

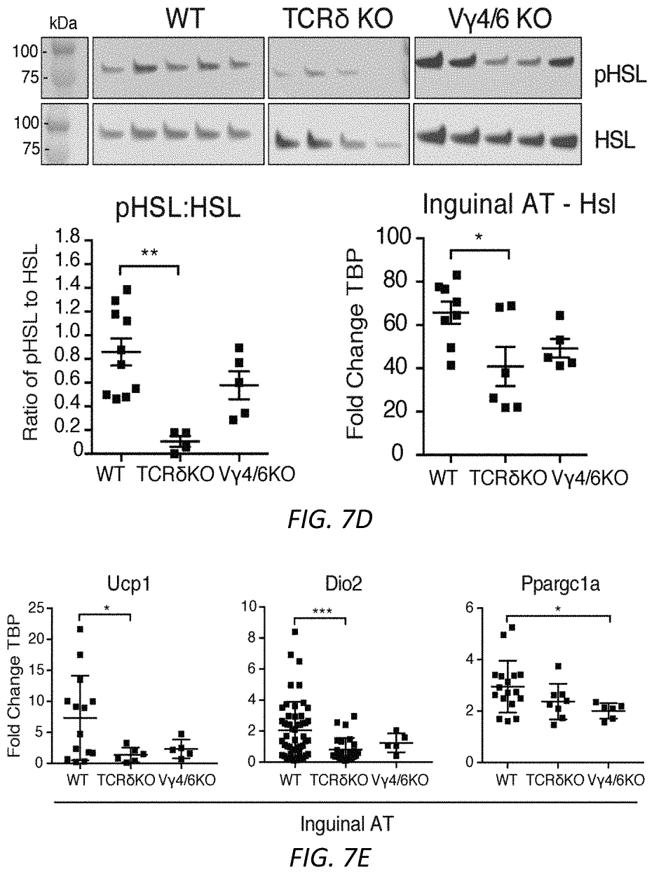

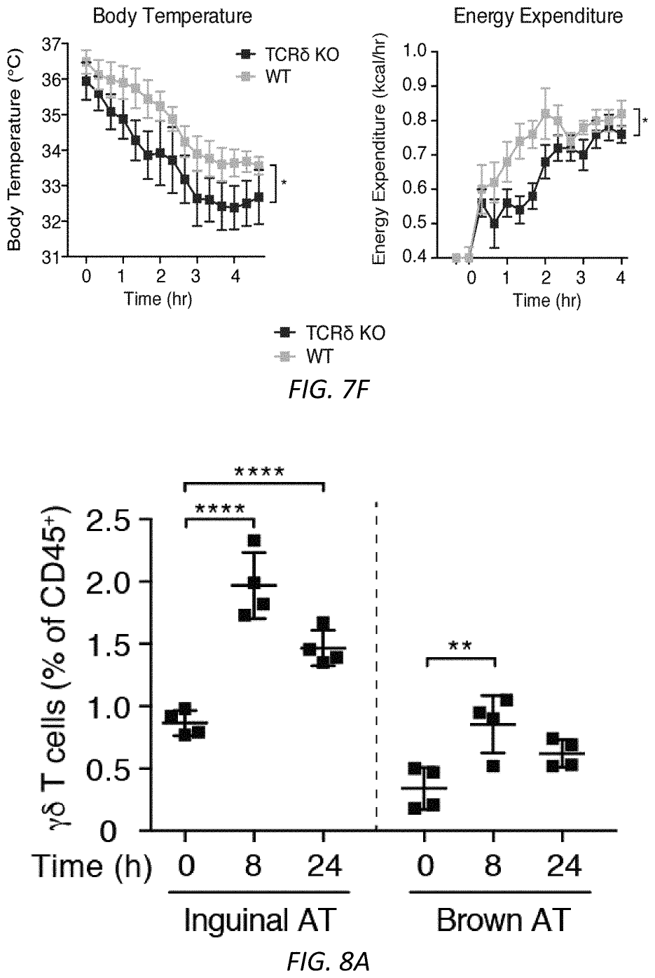

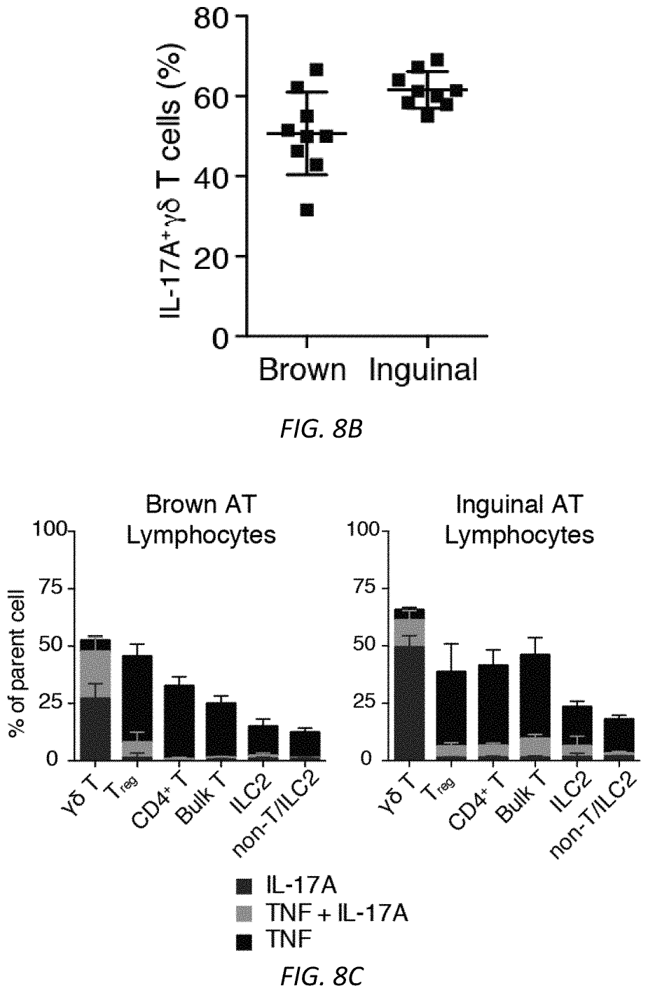

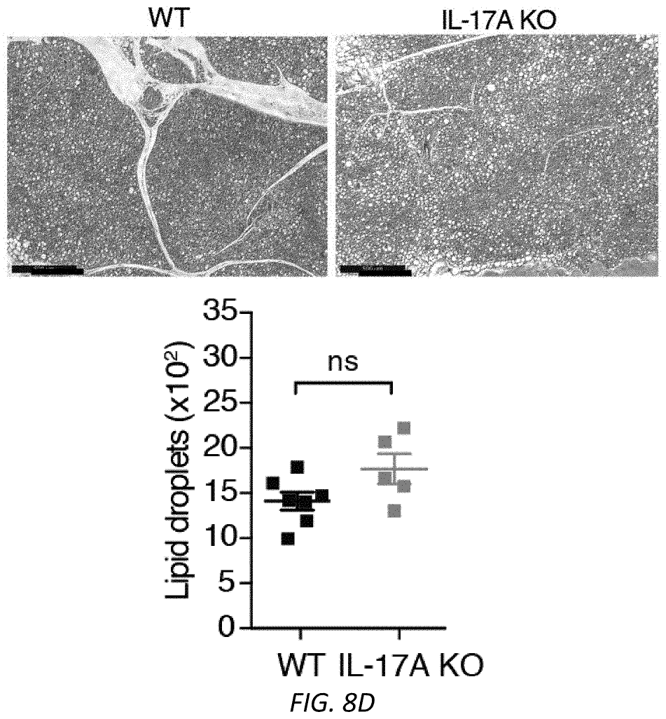

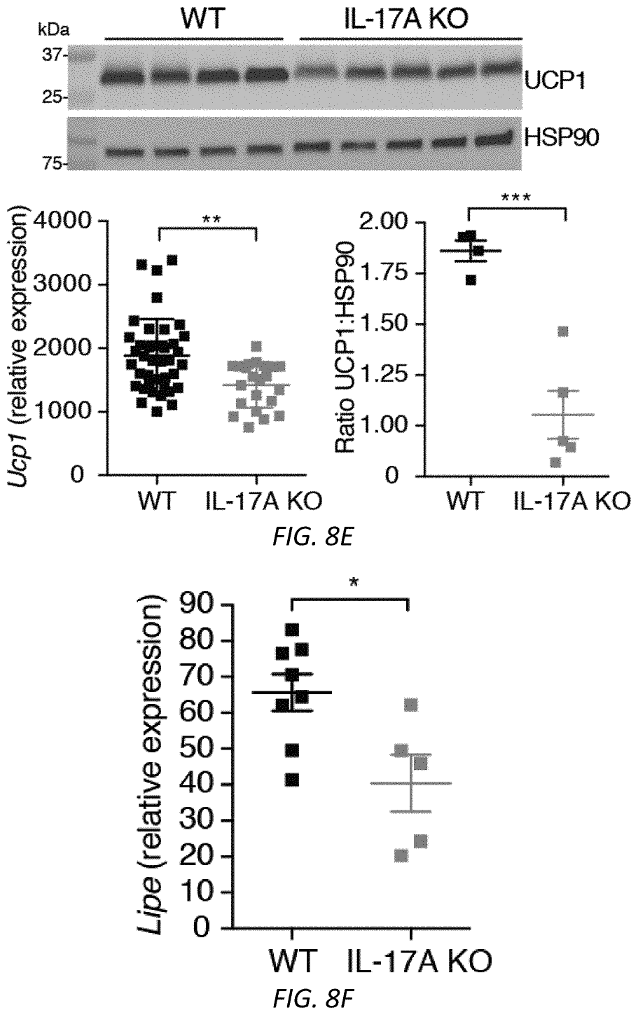

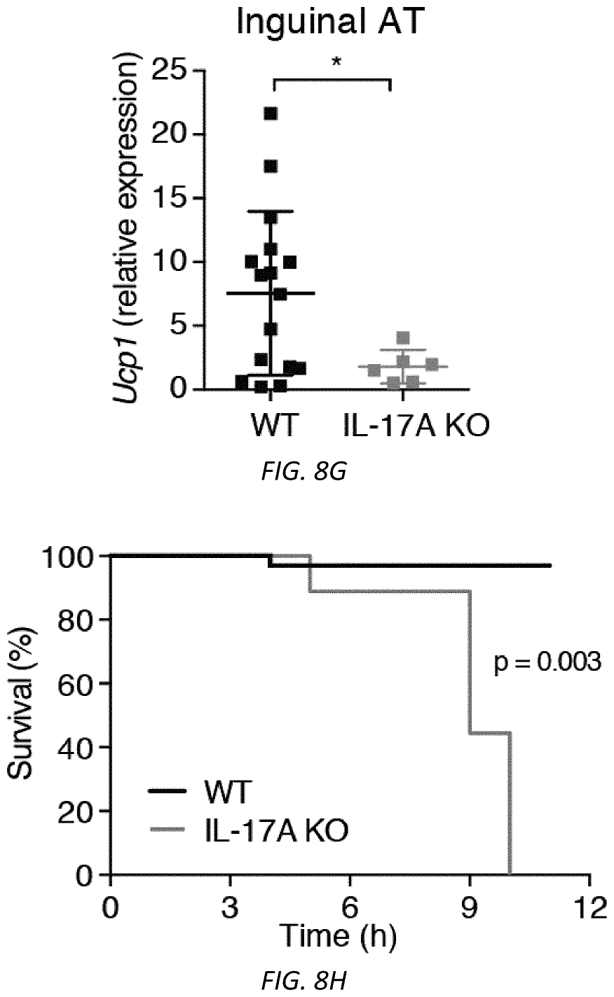

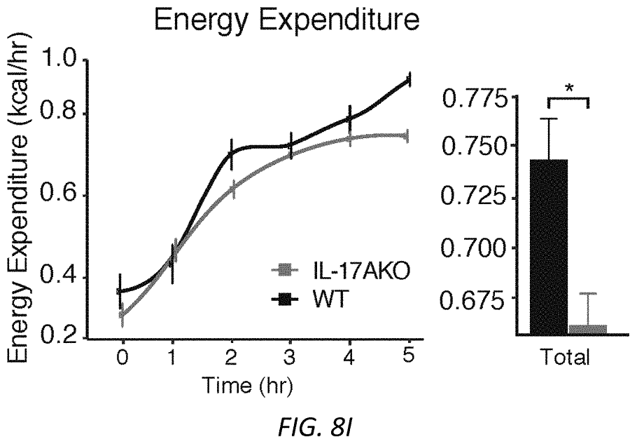

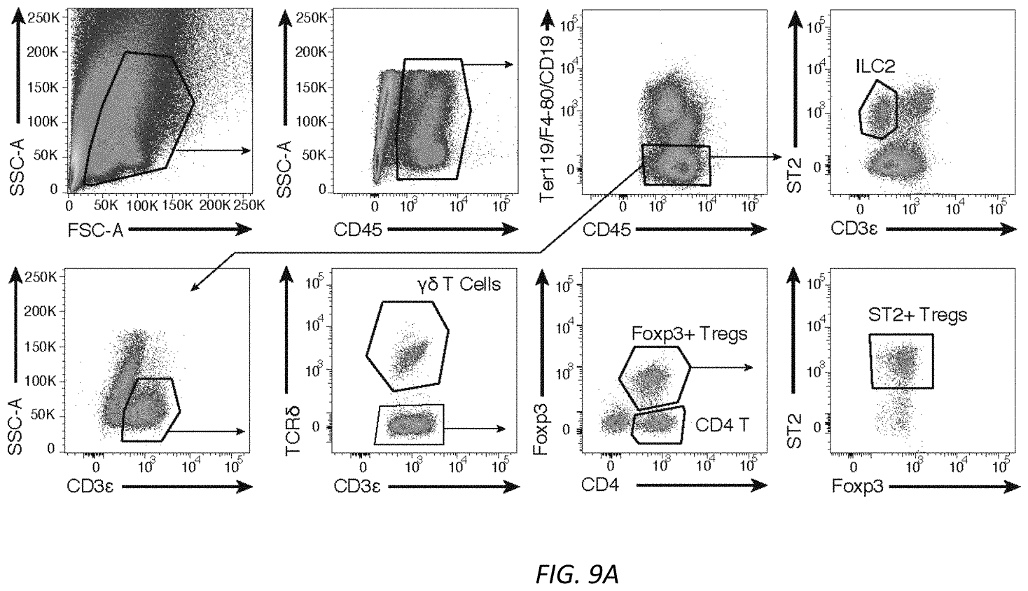

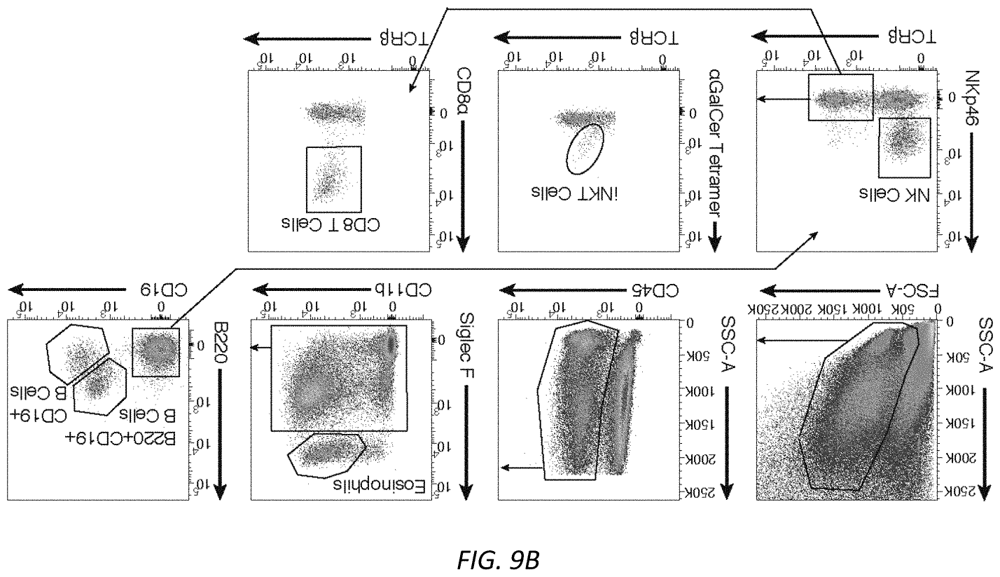

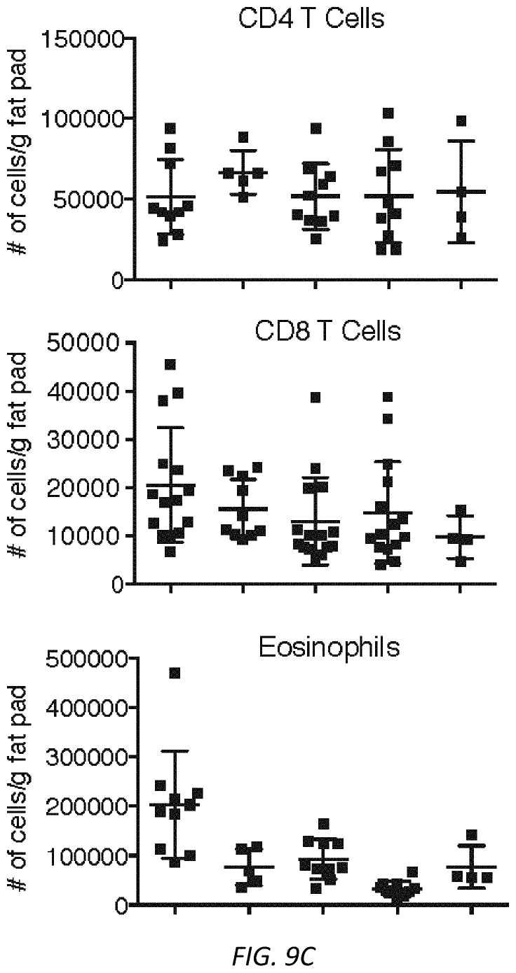

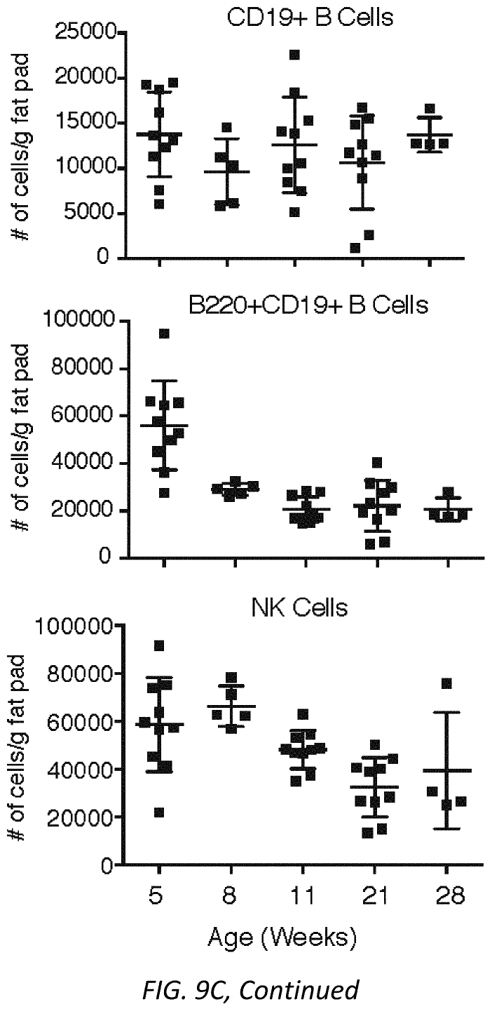

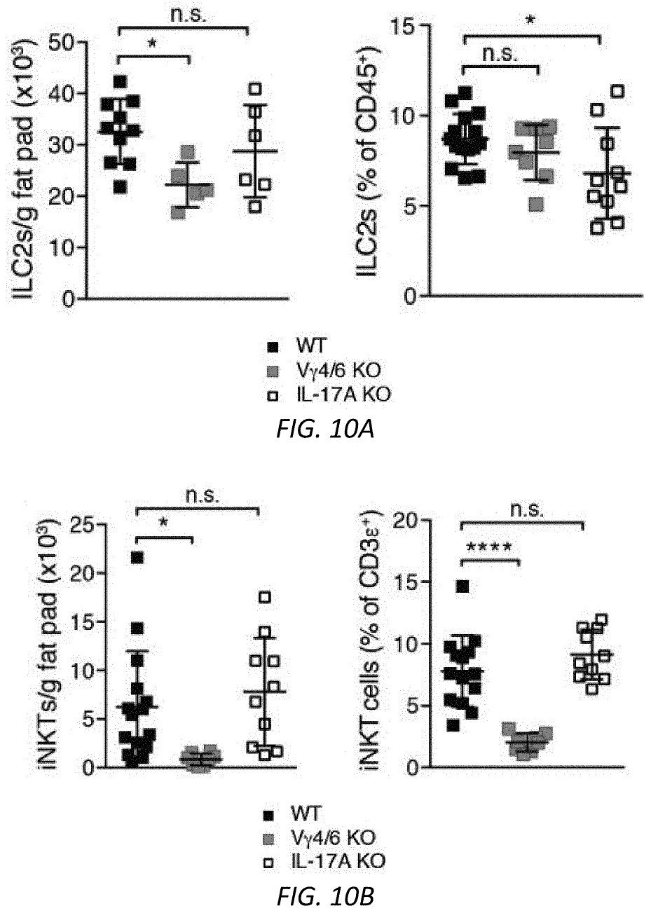

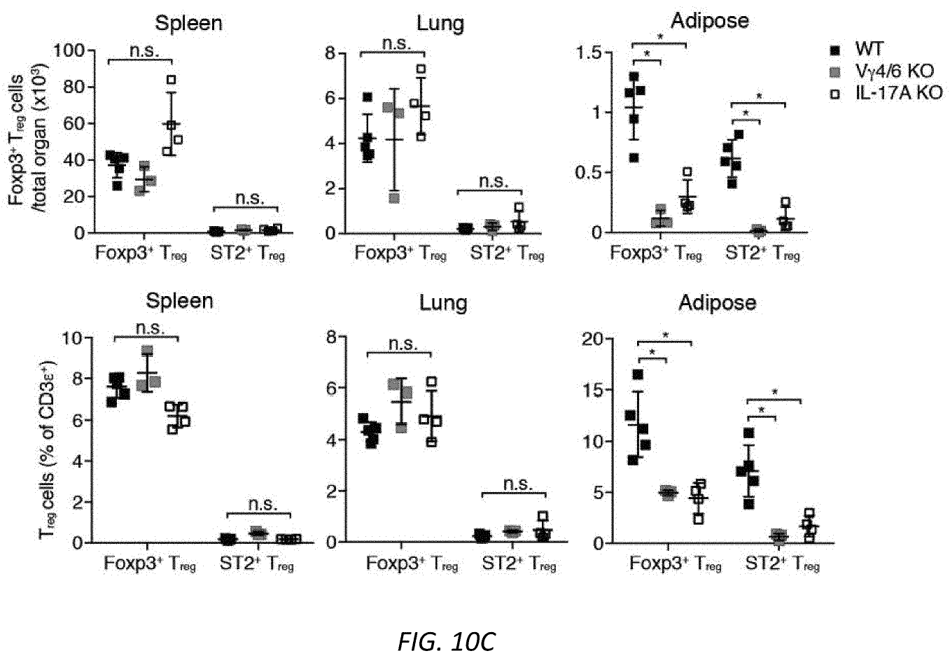

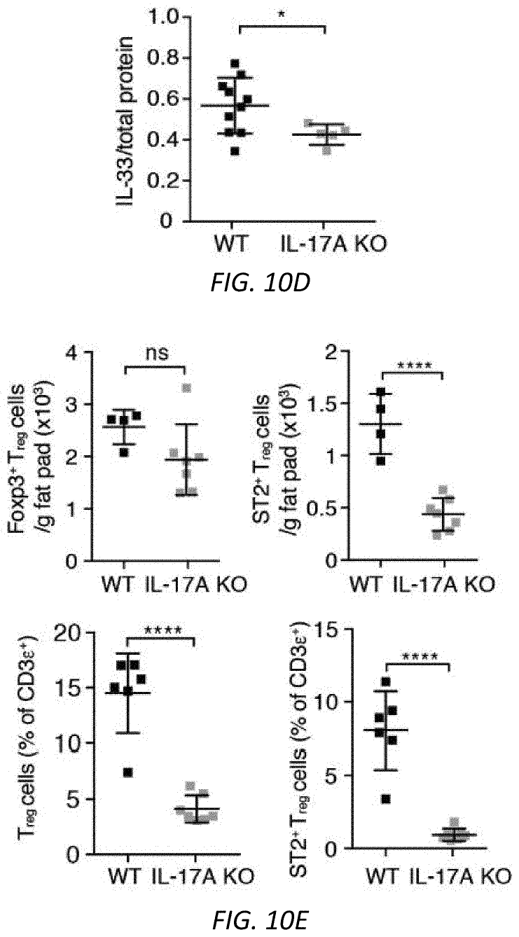

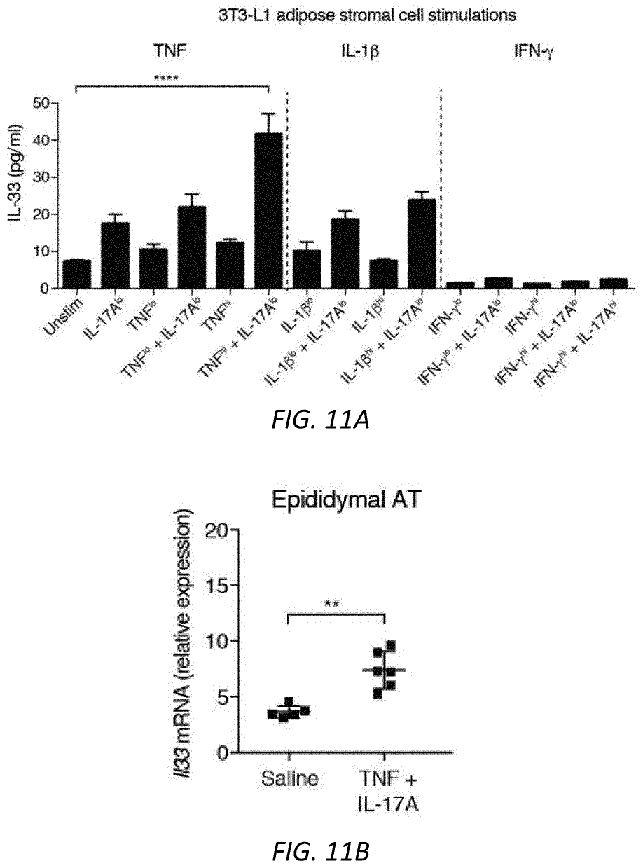

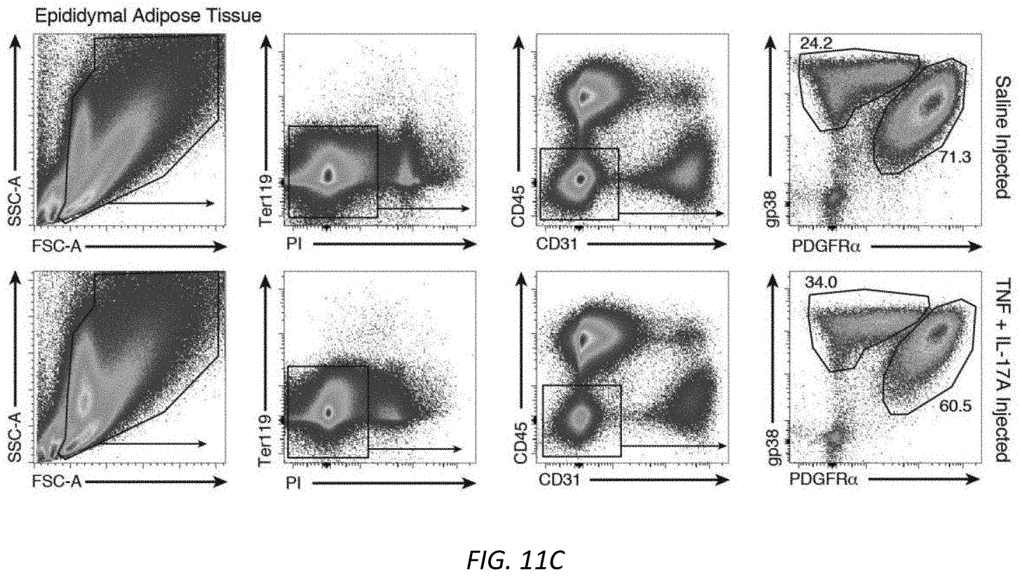

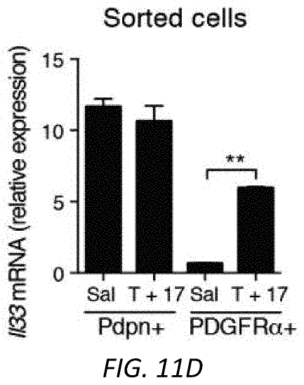

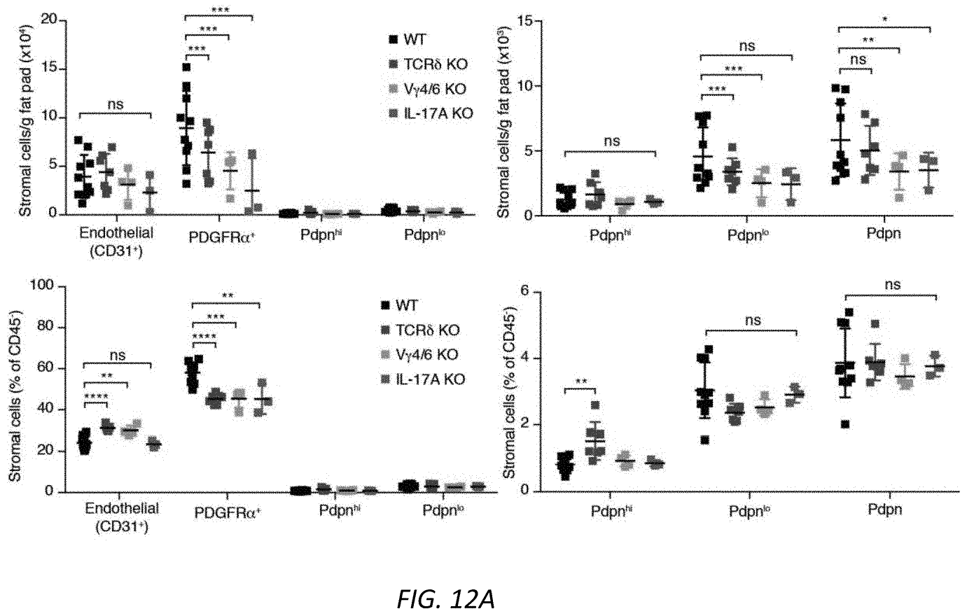

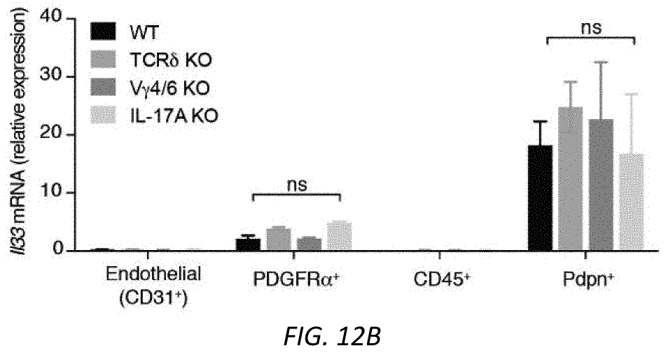

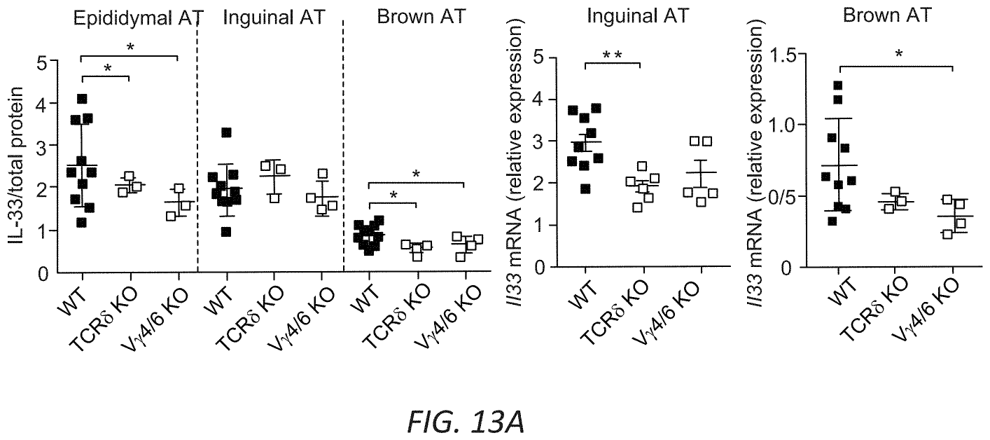

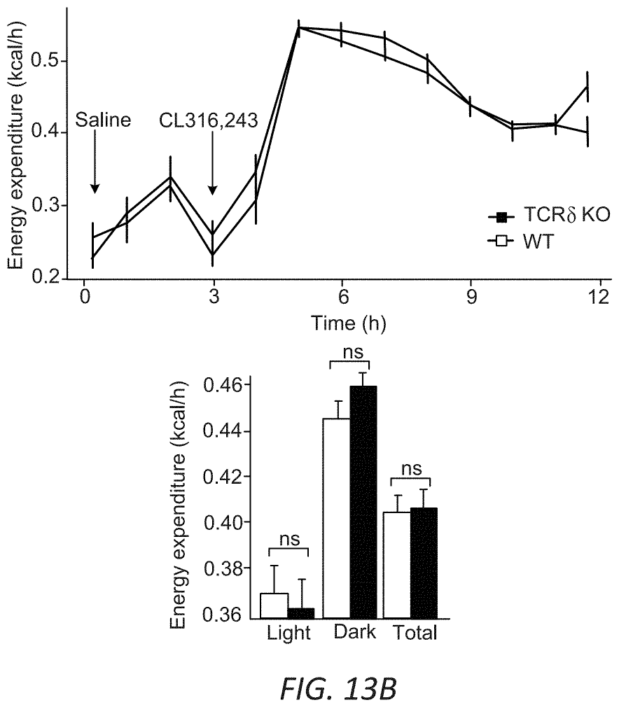

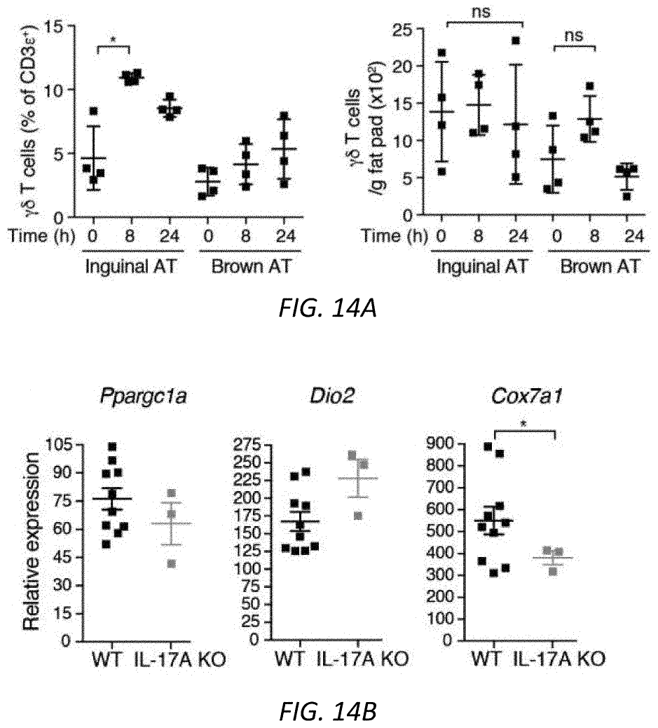

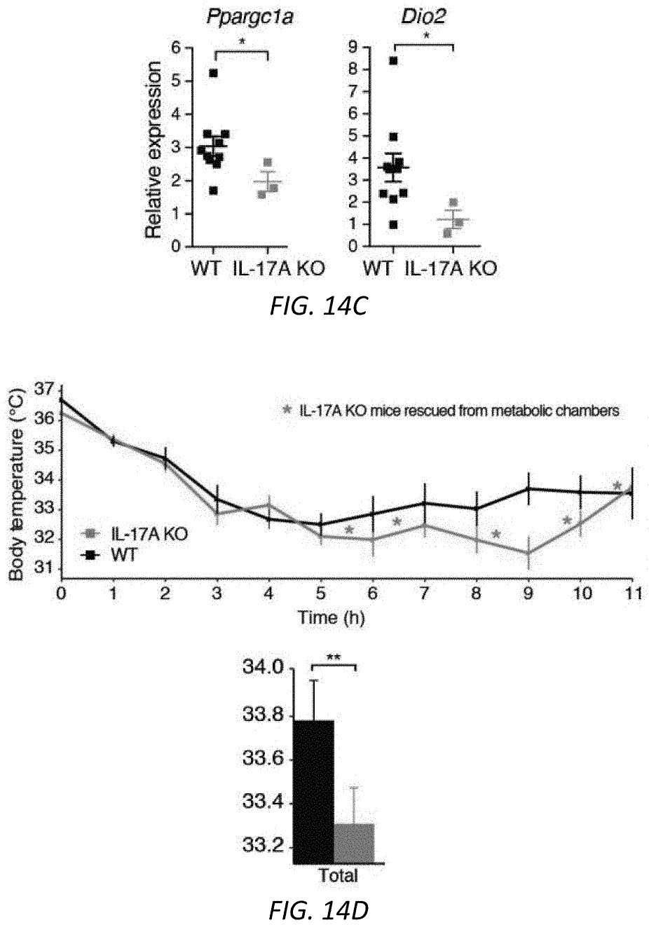

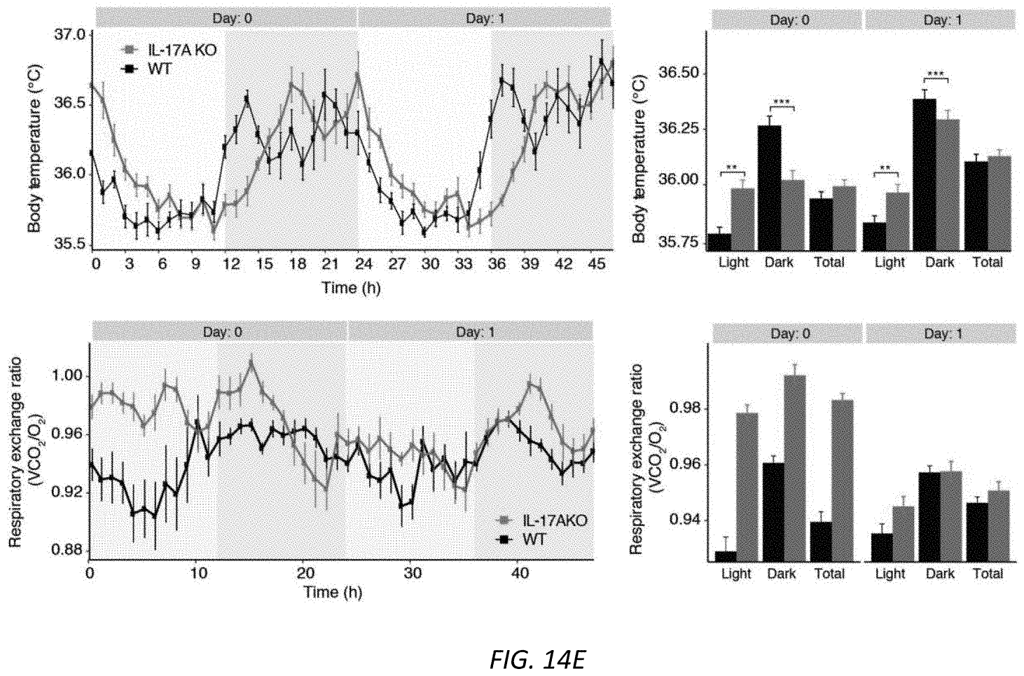

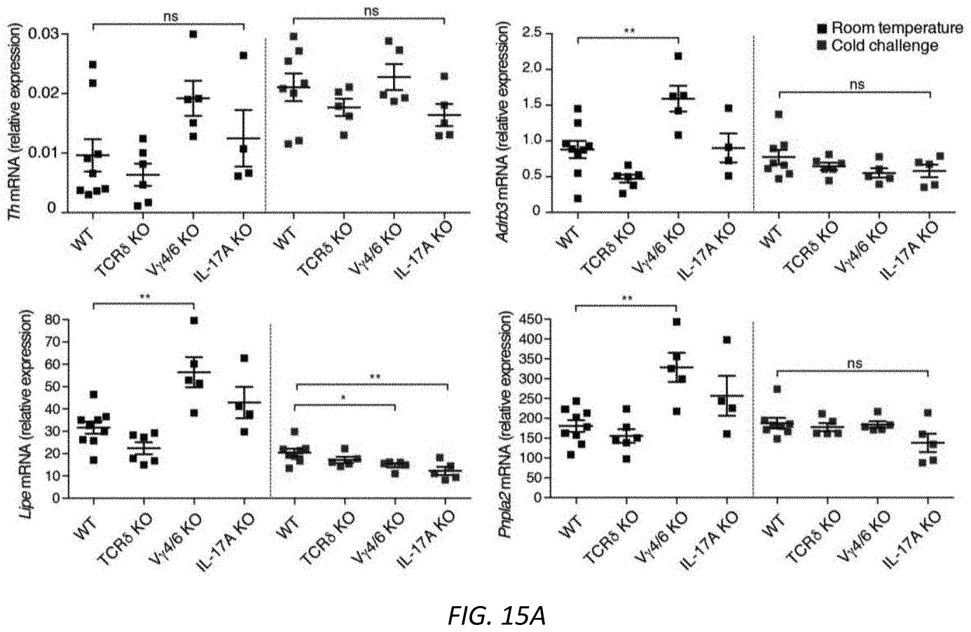

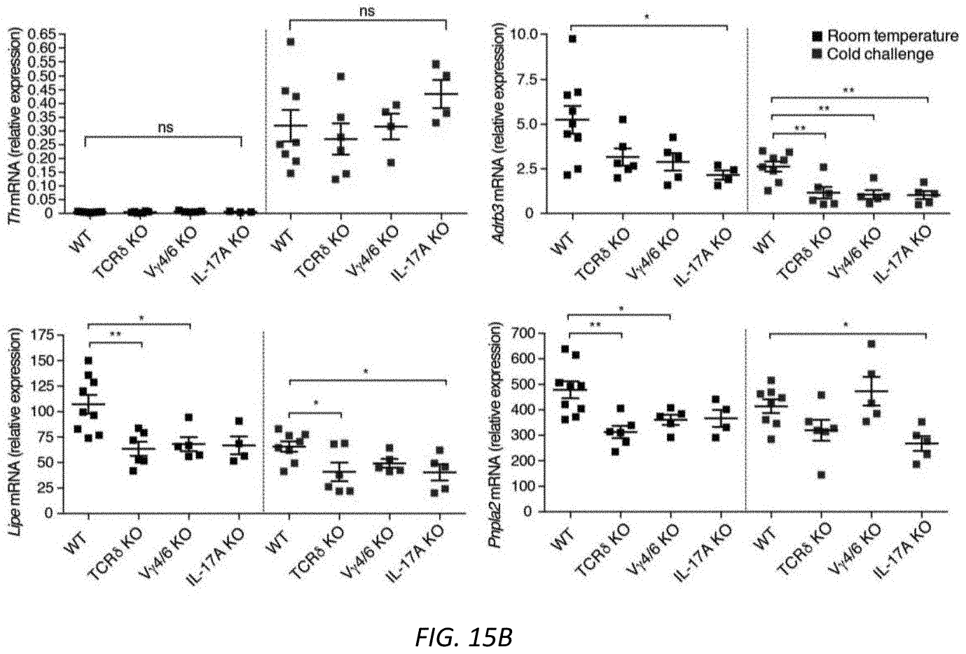

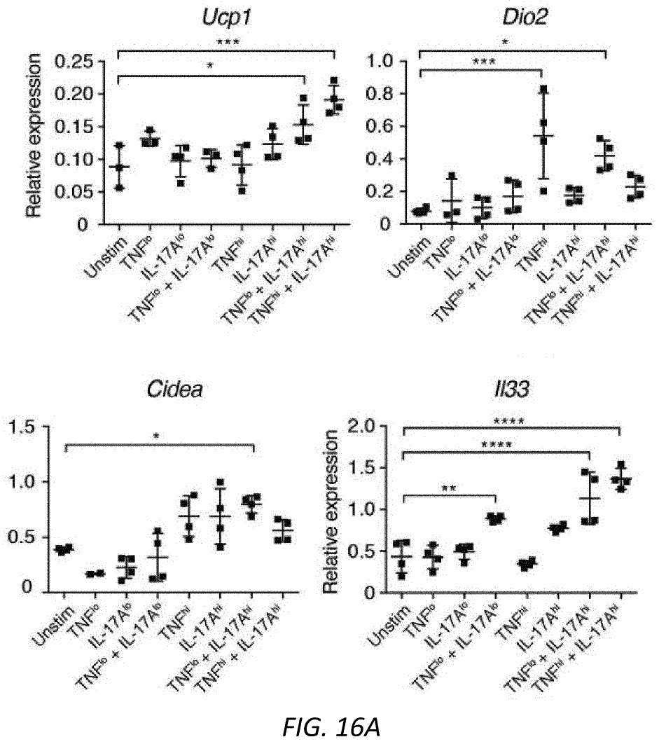

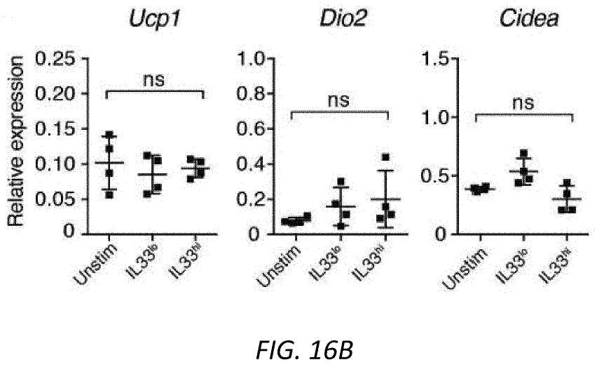

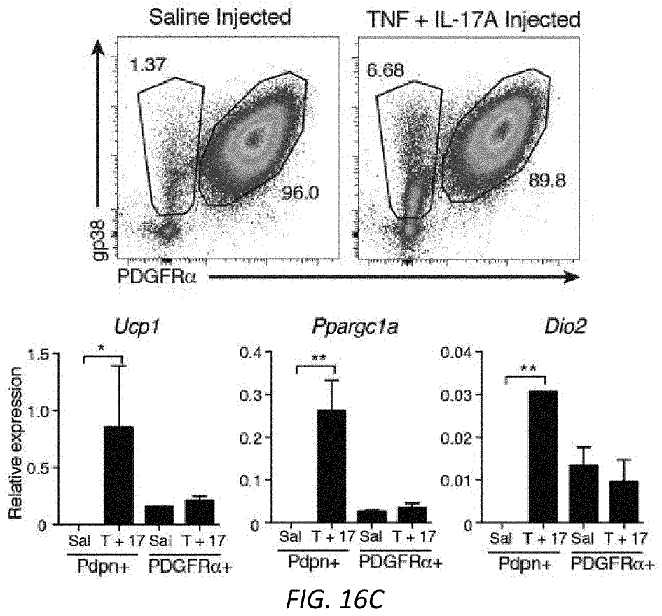

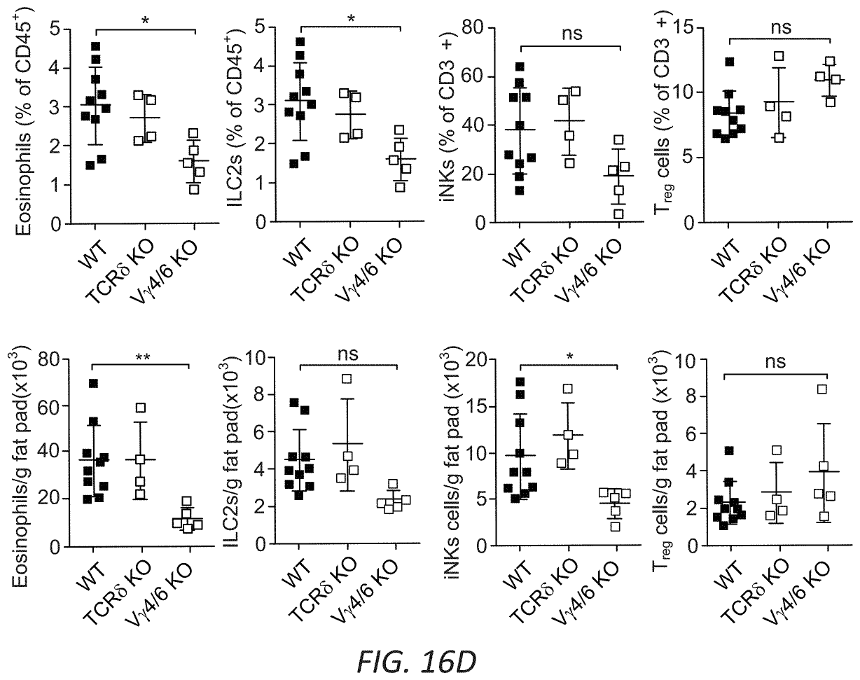

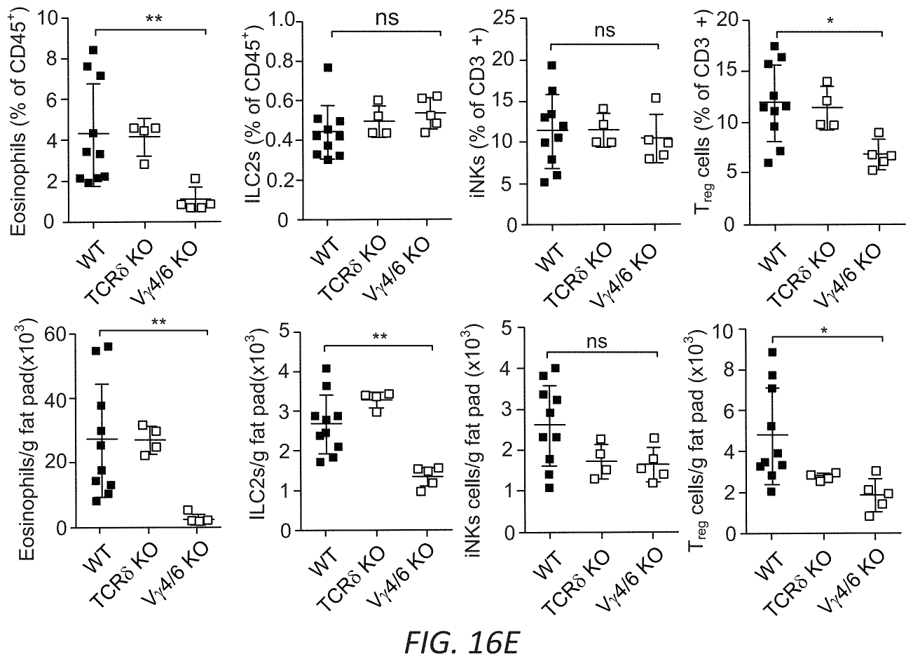

Next, we quantified the numbers of γδ T cells to determine changes in their frequency in adipose tissue over time. Interestingly, γδ T cells displayed similar accumulation kinetics as Foxp3⁺ T_{reg} cells in adipose tissue (FIG. 3A). In contrast, both ILC2s and iNKT cells, two populations previously shown to influence adipose T_{reg} numbers, decreased at the time of T_{reg} expansion (FIG. 3B). Besides T_{reg} cells and γδ T cells, no other lymphocyte population quantified increased with age (FIGS. 9A-9C). It is well described that T_{reg} cells expand in adipose tissue with age, but a unifying mechanism to explain their temporal accumulation remains unknown. Thus, we wondered if γδ T cells played a role in adipose T_{reg} homeostasis. To test this, we profiled wild-type and TCR6-deficient (Tcrd^{-/-}) mice and found that the frequency of T_{reg} cells was significantly reduced in Tcrd^{-/-} mice compared to wild-type counterparts after 20 weeks (FIG. 3C). A characteristic feature of adipose T_{reg} cells is their high expression of IL-10, KLRG1, and ST2¹⁰. T_{reg} cells sorted from Tcrd^{-/-} adipose tissue expressed significantly less Il10 (FIG. 3D) and surface KLRG1 compared to T_{reg} cells from wild-type mice (FIG. 3E). Lastly, we found a striking defect in ST2⁺ T_{reg} cell accumulation in Tcrd^{-/-} compared to wild-type littermates at 22 weeks of age, at the time of physiologic T_{reg} cell expansion (FIG. 3F). Together these results reveal a concomitant increase of γδ T cells and T_{reg} cells with age and the requirement of γδ T cells for visceral adipose T_{reg} accumulation.

### Example 4. PLZF⁺ γδ T Cells are Innate IL-17A Producing Cells

γδ T cells are generally recognized as innate-like lymphocytes that induce inflammation in response to pathogens and cellular stress. They rapidly secrete inflammatory cytokines such as TNF, interferon-γ (IFN-γ) and IL-17, as well as chemokines that recruit key phagocytes to injured or infected tissues²⁸. To understand how γδ T cells modulate T_{reg} numbers in adipose tissue, we further characterized their transcriptional phenotype and function. PLZF⁺ and PLZF⁻ γδ T cells were sorted from adipose tissue of Zbtb16^{GFP} mice for RNA sequencing and gene-expression analysis. Differential expression analysis showed 247 and 205 genes to be significantly upregulated in adipose PLZF⁺ and PLZF⁻ γδ T cells, respectively, of which the top 60 genes are shown (FIG. 4A). Consistent with the sorting strategy, Zbtb16 was among the most differential genes (FIGS. 4A,B). Notably, transcripts for genes including Sox13, Rorc, Il1r1, and Il23r were significantly upregulated in PLZF⁺ γδ T cells compared to PLZF⁻ counterparts (FIG. 4A,B). Moreover, the high transcript levels of Tcrg, Tcrd, Cd3e, and Cd3g in PLZF⁺ γδ T cells reaffirmed the flow cytometry data that distinguished CD3Σ^{hi} versus CD3Σ^{lo} γδ subsets (FIG. 4A). In contrast, PLZF⁻ γδ T cells showed significant expression of genes characteristic of NK cells including Ncr, Nkg7, Klrk1, Gzmb, and Gzma (FIG. 4A,B). These genes, combined with overexpression of genes encoding T-bet (Tbx21) and CD27 (Cd27), highlight their T_{H}1- and NK-like transcriptional phenotype (FIG. 4B). Lastly, we validated protein expression of RORγt and T-bet in γδ T cells and found them to be discretely expressed by PLZF⁺ and PLZF⁻ subsets, respectively (FIG. 4C).

Transcriptional analysis revealed that PLZF⁺ γδ T cells expressed Il17a, while PLZF⁻ γδ T cells expressed Ifng. As CD27 is useful to demarcate IL-17A-producing (CD27⁻) versus IFN-γ-producing (CD27⁺) γδ T cells, we used CD27 as a marker for functional analysis. Upon stimulation, PLZF⁺ γδ T cells exclusively produced IL-17A and TNF, while PLZF⁻ γδ T cells exclusively produced IFN-γ, consistent with their gene expression (FIG. 4D). Together, our transcriptional, phenotypic, and functional characterization highlight PLZF⁺ γδ T cells as innate IL-17Aproducing cells that exhibit a distinct effector program from the NK-like PLZF⁻ γδ T cells in adipose tissue.

### Example 5. ST2⁺ T_{reg} Numbers Depend on PLZF⁺ γδ T Cells and IL-17A

To parse the relative contributions of the two γδ T cell populations to the observed T_{reg} phenotype, we determined the kinetics of accumulation of the two subsets over time (FIG. 5A). Flow cytometry analysis of visceral fat pads showed IL-17A-producing PLZF⁺ γδ T cells, but not PLZF⁻ γδ T cells, expand with age (FIG. 5A). Importantly, profiling γδ T cells from Il17a^{GFP} mice showed that the numbers significantly increased in adipose tissue with age (FIG. 5B). We next asked if IL-17A was important for the observed T_{reg} cell accumulation. We found that IL-17A-deficient (Il17a^{-/-}) mice had significantly reduced numbers and frequencies of total Foxp3⁺ T_{reg} cells and failed to accumulate ST2⁺ T_{reg} cells in adipose tissue at 20 weeks of age (FIG. 5C). iNKT and ILC2 numbers were not different between wild-type and Il17a^{-/-} mice (FIGS. 10A-10E). This data suggests that IL-17A is a key factor to the homeostatic expansion of T_{reg} cells in visceral adipose tissue.

γδ T cells with specific V-gene rearrangements leave the thymus in concerted waves during neonatal development and seed tissues²⁹. The innate-IL17A producing subset is largely dominated by Vγ6⁺ TCRs, although other IL-17A-producing Vγ4⁺ cells can arise later. As some PLZF⁺ γδ T cells have been reported to harbor the canonical Vγ6⁺ TCR chain, we stained γδ T cells from adipose tissue with antibodies to determine TCR usage³⁰. At 20 weeks of age, the majority of CD3_{ε}^{hi}PLZF⁺CD27⁻ γδ T cells were Vγ6⁺, while CD3_{ε}^{lo}PLZF-CD27⁺ γδ T cells expressed Vγ1⁺ and Vγ4⁺ TCR chains and comprised a smaller fraction of total adipose γδ T cells (FIG. 5D).

To deplete the majority of PLZF⁺ γδ T cells, we characterized adipose tissue from Vγ4-Vγ6-deficient (Vg4/6^{-/-}) mice. We confirmed that Vg4/6^{-/-} mice had severely reduced numbers of PLZF⁺CD3ε^{hi} γδ T cells in adipose tissue, while PLZF⁻ CD3ε^{lo} γδ T cells were still present (FIG. 5E). Next, we analyzed 20-week-old wild-type and Vg4/6^{-/-} mice and found that, like Il17a^{-/-} and Tcrd^{-/-} mice, Vg4/6^{-/-} mice displayed significant reductions in total adipose Foxp3⁺ and ST2⁺ T_{reg} cells (FIG. 5F). When profiling other immune populations, we observed similar frequencies of ILC2s, but surprisingly, iNKT cells were significantly decreased in Vg4/6^{-/-} mice compared to wild-type, suggesting there may be crosstalk between PLZF⁺ γδ T cells and iNKT cells in adipose tissue (FIGS. 10A-10E). Interestingly, the reduced frequencies of ST2⁺ T_{reg} cells were not observed in spleen and lung of Il17a^{-/-} and Vg4/6^{-/-} mice (FIGS. 10A-10E). In summary, these data uncover an important role for both IL-17A and PLZF⁺ γδ T cells in age-dependent increases of adipose tissue T_{reg} cell numbers.

### Example 6. TNF and IL-17A Induce IL-33 in Adipose Stromal Cells

IL-33, a member of the IL-1 family of cytokines, is an important regulator of adipose ILC2 and T_{reg} cell homeostasis owing to the fact that both cell types express the cognate receptor, ST2^{11,15,31-34}. Adipose T_{reg} cells have high expression of ST2, and engagement of the ST2 receptor by IL-33 results in T_{reg} proliferation^{11,15}. We found that IL-33 protein increased with age in visceral adipose tissue, concomitant with T_{reg} and PLZF⁺ γδ T cell accumulation (FIG. 6A). Because of the significant reduction of T_{reg} numbers in Tcrd^{-/-}, Il17a^{-/-} and Vg4/6^{-/-} mice, we asked if γδ T cells affected IL-33 levels in adipose tissue. Indeed, Tcrd^{-/-} mice showed a significant decrease in IL-33 protein in adipose tissue but not spleen in 20-week-old mice (FIG. 6B). IL-2, a critical cytokine for T_{reg} maintenance in other peripheral organs was not different between wild-type and Tcrd^{-/-} mice (FIG. 6B). There was also a clear reduction in IL-33 protein in adipose tissue of Il17a^{-/-} and Vg4/6^{-/-} mice compared to wild-type (FIG. 6C). Interestingly, Il17a^{-/-} mice have significantly lower IL-33 protein and fewer ST2⁺ T_{reg} cell numbers in visceral adipose tissue even at 11 weeks of age, suggesting that this phenotype manifests earlier in Il17a^{-/-} mice compared to wild-type (FIGS. 10A-10E). Together, these results suggest that IL-17A-producing PLZF⁺ γδ T cells are necessary for age-induced increases IL-33 protein in visceral adipose tissue.

To assess the contribution of IL-17A producing PLZF⁺ γδ T cells to IL-33 concentrations, we sought to identify the IL-33 expressing cell type and importantly, test whether it would respond to IL-17A. Flow cytometry of the adipose stromal compartment identified a substantial population of CD31⁺ endothelial cells, as well as Pdpn⁺ (gp38⁺) stromal cells, among the non-hematopoietic (CD45⁻) pool (FIG. 6D). These Pdpn⁺ cells could further be segregated by their expression of the surface marker platelet-derived growth factor receptor-alpha (PDGFRα)(FIG. 6D), as well as CD26, CD9, and Cadherin-11 (Cdh11)(FIG. 6E). Staining for interleukin-17 receptor (IL-17R) to identify the subset that would respond to IL-17A identified Pdpn⁺CD26⁺PDGFRα⁻Cdh11⁺ stromal cells as the dominant IL-17R expressing population in adipose tissue (FIG. 6E). Intriguingly, when we quantified Il33 mRNA from sorted stromal cells, we found that Pdpn⁺PDGFRα⁻ cells highly expressed Il33 compared to other cells in adipose tissue (FIG. 6E). Our data highlighted Pdpn⁺CD26⁺PDGFRα⁻Cdh11⁺ stromal cells as the main IL-33 and IL-17R expressing cell type in visceral adipose tissue.

In addition to amplifying inflammation, IL-17A plays important homeostatic and anti-microbial roles at mucosal sites³⁵. However, its effects on adipose tissue stromal cells (fibroblasts and adipocytes), as well as other resident immune cells, are less appreciated36. Because Il17a^{-/-} mice exhibited severe decreases in T_{reg} cell numbers and IL-33 protein, we asked if IL-17A was sufficient to induce IL-33 expression in adipose stromal cells. Using 3T3L1 preadipocytes and fresh primary adipose stromal cells, we found that IL-17A stimulated Il33 mRNA and IL-33 protein, but it was the presence of both TNF and IL-17A that synergized to induce high expression of IL-33 (FIG. 6E). Moreover, this stimulation was specific for the combination of TNF and IL-17A, as IFN-γ or IL-1β failed to increase IL-33 to the same extent (FIGS. 11A-11D).

We next injected purified recombinant TNF and IL-17A into mice, and found that, upon injection, Il33 mRNA increased in adipose tissue (FIGS. 11A-11D). Mechanistically, TNF and IL-17A expanded IL-33 expressing Pdpn⁺ stromal cells and upregulated Il33 mRNA within the PDGFRα⁺ population (FIGS. 11A-11D). Although Pdpn⁺ stromal cells expressed the highest amounts of IL-33 at steady state, it appears that in vivo, both Pdpn⁺ and PDGFRα⁺ stromal subsets can contribute to endogenous IL-33 in visceral adipose tissue and can both be affected by TNF and IL-17A.

To assess why mice deficient in γδ T cells or IL-17A exhibited decreases in IL-33 in adipose tissue, we quantified the numbers and IL-33 expression of stromal populations from wild-type, Tcrd^{-/-}, Vg4/6^{-/-} and Il17a^{-/-} mice. We found that both Pdpn⁺ and PDGFRα⁺ stromal subset numbers were greatly decreased in Tcrd^{-/-}, Vg4/6^{-/-} and Il17^{-/-} mice, while IL-33 expression within stromal cells showed no significant differences (FIGS. 12A-12B). Our data suggest that in γδ-deficient and IL-17A-deficient animals, the decreased IL-33 owes largely to decreased numbers of IL-33 expressing or producing Pdpn⁺ and PDGFRα⁺ cells, respectively, and less so on lower Il33 mRNA within stromal populations.

Recent reports have demonstrated IL-33 expression in human adipocytes and endothelial cells^{37,38} and found that human omental T_{reg} cells express ST2¹¹. We asked whether this newly defined IL-17A-TNF-IL-33 axis existed in human adipose tissue. In human primary preadipocytes from both visceral and subcutaneous adipose depots, stimulation with TNF and IL-17A greatly enhanced expression of IL-33 (FIG. 6H). Taken together, TNF and IL-17A produced by PLZF⁺ γδ T cells promotes IL-33 within the stromal compartment in adipose tissue.

### Example 7. γδ T Cells and IL-17A Regulate Body Temperature

In addition to adipose immune regulation, IL-33 is important for thermogenesis, the metabolic adaptation to cold temperatures^{17,18}. When we extended our analysis to brown (BAT) and inguinal adipose tissue (iWAT), depots important for increasing body temperature, we found IL-33 protein to be significantly decreased in BAT of Tcrd^{-/-} and Vg4/6^{-/-} mice compared to wild-type (FIGS. 13A-13B). Although protein levels were not different in inguinal adipose tissue, Il33 mRNA was decreased in Tcrd^{-/-} and Vg4/6^{-/-} mice compared to wild-type (FIGS. 13A-13B), raising the possibility that mice lacking γδ T cells might exhibit defects in body temperature regulation.

In support, histological sections of BAT from cold challenged mice showed Tcrd^{-/-} and Vg4/6^{-/-} mice to harbor more lipids in brown adipocytes compared to wild-type littermates (FIG. 7A). Importantly, Tcrd^{-/-} and Vg4/6^{-/-} mice had decreased UCP1 protein in BAT, and lower expression of thermogenic genes including Ppargc1a, Dio2, and Cox7a1 compared to wild-type after cold challenge (FIG. 7B,C). Remarkably, when we evaluated iWAT after cold challenge, Tcrd^{-/-} and Vg4/6^{-/-} mice were unable to burn lipids evidenced by gross anatomy (FIGS. 13A-13B), and had decreased expression and activation of hormone sensitive lipase (HSL), a critical lipolysis enzyme (FIG. 7D). Like brown adipose tissue, the inguinal depot from Tcrd^{-/-} and Vg4/6^{-/-} mice after cold challenge showed decreased expression of thermogenic genes (FIG. 7E), suggesting mice deficient in γδ T cells are unable to upregulate adaptive thermogenesis in BAT and iWAT.

To determine whether these local adipose defects had physiological consequences, we measured body temperature and energy expenditure by indirect calorimetry. Tcrd^{-/-} mice dropped body temperature significantly more rapidly than wild-type in the cold and correspondingly were unable to increase energy expenditure (FIG. 7F). Of note, when challenged with the β-adrenergic agonist CL-316 243, which maximally activates BAT thermogenesis, Tcrd^{-/-} mice did not show a defect in their ability to expend energy as heat, suggesting that β-adrenergic signaling was to downstream of γδ T cell control (FIGS. 13A-13B). Thus, mice deficient in γδ T cells were unable to engage non-shivering thermogenesis in response to cold, in part due to an inability to upregulate factors important for turning on the thermogenic program in BAT and iWAT.

To better understand how γδ T cells promoted thermogenic responses, we quantified the frequency and cytokine profile of γδ T cells in BAT and iWAT after cold challenge. We found that 8 hours after cold challenge, γδ T cells significantly increased in frequency in both BAT and iWAT and remained elevated at 24 hours (FIG. 8A). Despite their increased frequency, the number of γδ T cells did not change, suggesting the decline of other immune populations with cold (FIGS. 14A-14D). When γδ T cells were stimulated with PMA and ionomycin, greater than 40% produced IL-17A in BAT and iWAT (FIG. 8B). Conversely, other immune populations produced little IL-17A, highlighting the role of γδ T cells as the dominant source of IL-17A in both depots (FIG. 8C). γδ T cells thus become a larger percentage of immune cells after cold challenge and are a major source of IL-17A in situ.

As γδ T cells produce IL-17A in BAT and iWAT, we hypothesized that IL-17A might be an important regulator of the thermogenic phenotype observed. Indeed, like Tcrd^{-/-} and Vg4/6^{-/-} mice, the BAT of Il17a^{-/-} mice contained more lipid droplets (FIG. 8D) and failed to upregulate UCP1 (FIG. 8E) compared to wild-type after cold. Moreover, defects in lipolysis were observed in the iWAT of Il17a^{-/-} mice (FIG. 8F), and a similar inability of Il17a^{-/-} mice to upregulate Ucp1 in iWAT was measured (FIG. 8G). Il17a^{-/-} mice showed decreased expression of other thermogenic genes in both BAT and iWAT (FIGS. 14A-14D). Strikingly, when WT and Il17a^{-/-} mice were placed in metabolic cages for indirect calorimetry assessment, all of the Il17a^{-/-} mice had to be rescued from death 5-12 hours after cold challenge because of their inability to increase energy expenditure (FIG. 8H,L). This is evidenced by their inability to increase body temperature 5 hours after cold challenge (FIGS. 14A-14D). Interestingly, Il17a^{-/-} mice also showed abnormal circadian control of body temperature and respiratory exchange ratio (RER) (FIGS. 14A-14D). Together, these data support a critical role for IL-17A in body temperature regulation.

To understand the mechanism behind thermogenic control by γδ T cells and IL-17A, we first quantified gene expression for thermogenic enzymes and receptors (FIGS. 15A-15B). Interestingly, Adrb3 mRNA was decreased across all genotypes in iWAT at thermoneutrality and after cold. Lipe and Pnpla2, two key genes of lipolysis, were also significantly decreased, suggesting mice lacking γδ T cells or IL-17A were less sensitive to catecholamine stimulation for lipolysis induction. Second, stimulation with TNF and IL-17A synergized to upregulate thermogenic genes including Ucp1, Dio2, Cidea, and Il33 in brown adipocyte cultures (FIGS. 15A-15B). Stimulation with IL-33 itself did not induce the same increases in gene expression, suggesting that TNF and IL-17A can induce a thermogenic program in BAT independent of IL-33 (FIGS. 16A-16C). In iWAT, TNF and IL-17A injections in vivo were sufficient to induce Ucp1, Ppargc1a, and Dio2 in sorted Pdpn⁺ stromal cells, but not PDGFRα⁺ cells (FIGS. 16A-16C). Lastly, we quantified BAT and iWAT of wild-type, Tcrd^{-/-} and Vγ4/6^{-/-} mice for immune cells known to be important for thermogenic responses (FIGS. 16A-16C). There were no differences in ILC2, eosinophil, iNKT, and Treg frequencies between wild-type and Tcrd^{-/-} mice in both depots, suggesting the thermogenic defects seen in γδ T cell deficient mice were independent of other immune populations. Together, our data suggest γδ T cells can promote thermogenic responses directly through the cytokines they produce, namely TNF and IL-17A, and indirectly through maintenance of catecholamine sensitivity.

### REFERENCES

1. Brestoff, J. R. & Artis, D. Immune regulation of metabolic homeostasis in health and disease. Cell 161, 146-160 (2015).

2. Kohlgruber, A. & Lynch, L. Adipose tissue inflammation in the pathogenesis of type 2 diabetes. Curr. Diab. Rep. 15, 92; 1-11 (2015).

3. Lynch, L. et al. Regulatory iNKT cells lack expression of the transcription factor PLZF and control the homeostasis of T(reg) cells and macrophages in adipose tissue. Nat. Immunol. 16, 85-95 (2015).

4. Lynch, L. et al. Adipose tissue invariant NKT cells protect against diet-induced obesity and metabolic disorder through regulatory cytokine production. Immunity 37, 574-587 (2012).

5. Molofsky, A. B. et al. Innate lymphoid type 2 cells sustain visceral adipose tissue eosinophils and alternatively activated macrophages. Journal of Experimental Medicine 210, 535-549 (2013).

6. Nussbaum, J. C. et al. Type 2 innate lymphoid cells control eosinophil homeostasis. Nature 502, 245-248 (2013).

7. Lynch, L. et al. iNKT Cells Induce FGF21 for Thermogenesis and Are Required for Maximal Weight Loss in GLP1 Therapy. Cell Metab. 24, 510-519 (2016).

8. Brestoff, J. R. et al. Group 2 innate lymphoid cells promote beiging of white adipose tissue and limit obesity. Nature 519, 242-246 (2015).

9. Feuerer, M. et al. Lean, but not obese, fat is enriched for a unique population of regulatory T cells that affect metabolic parameters. Nat. Med. 15, 930-939 (2009).

10. Cipolletta, D. et al. PPAR-γ is a major driver of the accumulation and phenotype of adipose tissue Treg cells. Nature 486, 549-553 (2012.

11. Vasanthakumar, A. et al. The transcriptional regulators IRF4, BATF and IL-33 orchestrate development and maintenance of adipose tissue-resident regulatory T cells. Nat. Immunol. 16, 276-285 (2015).

12. Bapat, S. P. et al. Depletion of fat-resident Treg cells prevents age-associated insulin resistance. Nature 528, 137-141 (2015).

13. Cipolletta, D. et al. Adipose tissue-resident regulatory T cells: phenotypic specialization, functions and therapeutic potential. Immunology 142, 517-525 (2014).

14. Panduro, M. et al. Tissue Tregs. Annu. Rev. Immunol. 34, 609-633 (2016).

15. Kolodin, D. et al. Antigen- and cytokine-driven accumulation of regulatory T cells in visceral adipose tissue of lean mice. Cell Metab. 21, 543-557 (2015).

16. Molofsky, A. B. et al. Interleukin-33 and Interferon-γ Counter-Regulate Group 2 Innate Lymphoid Cell Activation during Immune Perturbation. Immunity 43, 161-174 (2015).

17. Mathis, D. IL-33, Imprimatur of Adipocyte Thermogenesis. Cell 166, 794-795 (2016).

18. Odegaard, J. I. et al. Perinatal Licensing of Thermogenesis by IL-33 and ST2. Cell 166, 841-854 (2016).

19. Lee, M.-W. et al. Activated type 2 innate lymphoid cells regulate beige fat biogenesis. Cell 160, 74-87 (2015).

20. Jackson-Jones, L. H. et al. Fat-associated lymphoid clusters control local IgM secretion during pleural infection and lung inflammation. Nat. Commun. 7, 12651 (2016).

21. Pichery, M. et al. Endogenous IL-33 is highly expressed in mouse epithelial barrier tissues, lymphoid organs, brain, embryos, and inflamed tissues: in situ analysis using a novel Il-33 -LacZ gene trap reporter strain. The Journal of Immunology 188, 3488-3495 (2012).

22. Paget, C. et al. CD3bright signals on γδ T cells identify IL-17A-producing Vγ6Vγ1+ T cells. Immunol. Cell Biol. 93, 198-212 (2015).

23. Ribot, J. C. et al. CD27 is a thymic determinant of the balance between interferon-gamma- and interleukin 17-producing gammadelta T cell subsets. Nat. Immunol. 10, 427-436 (2009).

24. Kreslaysky, T. et al. TCR-inducible PLZF transcription factor required for innate phenotype of a subset of gammadelta T cells with restricted TCR diversity. Proc. Natl. Acad. Sci. U.S.A. 106, 12453-12458 (2009).

25. Lu, Y. et al. PLZF Controls the Development of Fetal-Derived IL-17+Vγ6+γδ T Cells. The Journal of Immunology 195, 4273-4281 (2015).

26. Savage, A. K. et al. The transcription factor PLZF directs the effector program of the NKT cell lineage. Immunity 29, 391-403 (2008).

27. Alonzo, E. S. & Sant'Angelo, D. B. Development of PLZF-expressing innate T cells. Current Opinion in Immunology 23, 220-227 (2011).

28. Vantourout, P. & Hayday, A. Six-of-the-best: unique contributions of γδ T cells to immunology. Nat. Rev. Immunol. 13, 88-100 (2013).

29. Wei, Y.-L. et al. A Highly Focused Antigen Receptor Repertoire Characterizes γδ T Cells That are Poised to Make IL-17 Rapidly in Naive Animals. Front. Immunol. 6, 118 (2015).

30. Roark, C. L. et al. Subset-specific, uniform activation among Vγ6/Vδ1+γδ T cells elicited by inflammation. Journal of Leukocyte Biology 75, 68-75 (2004).

31. Van Dyken, S. J. et al. Chitin activates parallel immune modules that direct distinct inflammatory responses via innate lymphoid type 2 and γδ T cells. Immunity 40, 414-424 (2014).

32. Vannella, K. M. et al. Combinatorial targeting of TSLP, IL-25, and IL-33 in type 2 cytokine-driven inflammation and fibrosis. Science Translational Medicine 8, 337ra65 (2016).

33. Schiering, C. et al. The alarmin IL-33 promotes regulatory T-cell function in the intestine. Nature 513, 564-568 (2014).

34. Kuswanto, W. et al. Poor Repair of Skeletal Muscle in Aging Mice Reflects a Defect in Local, Interleukin-33-Dependent Accumulation of Regulatory T Cells. Immunity 44, 355-367 (2016).

35. Hepworth, M. R. Innate Lymphoid Cell Regulation: Meeting the Long-Lost Cousin. Trends Immunol. 38, 873-874 (2017).

36. ZAlliga, L. A. et al. IL-17 regulates adipogenesis, glucose homeostasis, and obesity. The Journal of Immunology 185, 6947-6959 (2010).

37. Wood, I. S. et al. IL-33 , a recently identified interleukin-1 gene family member, is expressed in human adipocytes. Biochem. Biophys. Res. Commun. 384, 105-109 (2009).

38. Zeyda, M. et al. Severe obesity increases adipose tissue expression of interleukin-33 and its receptor ST2, both predominantly detectable in endothelial cells of human adipose tissue. Int J Obes (Load) 37, 658-665 (2013). 39. Martin, N. T. & Martin, M. U. Interleukin 33 is a guardian of barriers and a local alarmin. Nat. Immunol. 17, 122-131 (2016).

40. Kovalovsky, D. et al. The BTB-zinc finger transcriptional regulator PLZF controls the development of invariant natural killer T cell effector functions. Nat. Immunol. 9, 1055-1064 (2008).

41. Wagers, A. J. et al. Little evidence for developmental plasticity of adult hematopoietic stem cells. Science 297, 2256-2259 (2002).

42. Okada, K. et al. Thioesterase superfamily member 1 suppresses cold thermogenesis by limiting the oxidation of lipid droplet-derived fatty acids in brown adipose tissue. Mol Metab 5, 340-351 (2016).

43. Kim, D., Langmead, B. & Salzberg, S. L. HISAT: a fast spliced aligner with low memory requirements. Nature Methods 12, 357-360 (2015).

44. Li, H. et al. The Sequence Alignment/Map format and SAMtools. Bioinformatics 25, 2078-2079 (2009).

45. Trapnell, C. et al. Transcript assembly and quantification by RNA-Seq reveals unannotated transcripts and isoform switching during cell differentiation. Nat. Biotechnol. 28, 511-515 (2010).

46. Trapnell, C. et al. Differential gene and transcript expression analysis of RNA-seq experiments with TopHat and Cufflinks. Nat Protoc 7, 562-578 (2012).

47. Trapnell, C. et al. Differential analysis of gene regulation at transcript resolution with RNA-seq. Nat. Biotechnol. 31, 46-53 (2013).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-7. (canceled)

8. A method of inhibiting weight loss in a subject with cachexia, the method comprising administering to the subject therapeutically effective amounts of a combination of agents comprising:
(i) an inhibitor of Tumor Necrosis Factor alpha (TNFα) or the TNF Receptor (TNFR), and
(ii) an inhibitor of Interleukin 17 (IL-17) or the IL-17 Receptor (IL-17R).

9. (canceled)

10. The method of claim 8, wherein the subject has a chronic illness or cancer.

11. The method of claim 10, wherein the cancer is not IL-17 dependent.

12. The method of claim 10, wherein the cancer is pancreatic cancer.

13. The method of claim 10, wherein the subject has COPD, diabetic kidney disease, or heart failure.

14. The method of claim 8, wherein the agents are modified to increase half-life.

15. The method of claim 8, wherein the agents are targeted to adipose tissue.

16. The method of claim 8, wherein the inhibitor of IL-17 or the IL-17R is selected from the group consisting of secukinumab, ustekinumab, brodalumab and ixekizumab.

17. The method of claim 8, wherein the inhibitor of TNFα or the TNFR is selected from the group consisting of infliximab, adalimumab, certolizumab pegol, golimumab, and etanercept.

18. The method of claim 8, wherein the inhibitor of TNFα or the TNFR and the inhibitor of IL-17 or the IL-17R are administered in a single composition.

19. The method of claim 8, wherein the inhibitor of TNFα or the TNFR and the inhibitor of IL-17 or the IL-17R are administered in separate compositions.
